(19) United States
(12) Patent Application Publication
(10) Pub. No.: US 2008/0166375 A1
(43) Pub. Date: Jul. 10, 2008

(54) Activation of Recombinant Diphtheria Toxin Fusion Proteins by Specific Proteases Highly Expressed on the Surface of Tumor Cells

(75) Inventors: Stephen H. Leppla, Bethesda, MD (US); Jennifer Avallone, Flemington, NJ (US); Thomas Bugge, Bethesda, MD (US); Shi-Hui Liu, Gaithersburg, MD (US); Manuel Osorio, Bethesda, MD (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: The Government of the United States of America, as rep. by the Secretary of Health and Human Service, Rockville, MD (US)

(21) Appl. No.: 10/554,076

(22) PCT Filed: May 6, 2004
(86) PCT No.: PCT/US04/14306

§ 371 Date: May 29, 2007

Related U.S. Application Data
(60) Provisional application No. 60/468,577, filed on May 6, 2003.

(51) Int. Cl. A61K 39/02; C07H 21/04; C12N 15/00; C07K 16/00; C12N 5/00

(52) U.S. Cl. 424/236.1; 536/23.4; 435/320.1; 530/350; 435/325

(57) ABSTRACT

The present invention provides compositions and methods for inhibiting abnormal cell growth. In particular, the invention provides nucleic acids encoding Diphtheria toxin fusion proteins comprising residues 1-388 of Diphtheria toxin, wherein the native furin cleavage site has been substituted for a matrix metalloproteinase or plasminogen activator cleavage site, and a heterologous polypeptide and the polypeptides encoded by such nucleic acids. In addition, the invention provides methods of treating cancer by administering such polypeptides.

### CROSS-REFERENCES TO RELATED APPLICATIONS

This application claims the benefit of U.S. Provisional Application No. 60/468,577, filed May 6, 2003, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK

Not applicable.

### BACKGROUND OF THE INVENTION

Despite enormous investments of financial and human resources, cancer remains one of the major causes of death. For example, multiple hematological malignancies (e.g., adult and pediatric acute myeloid leukemia (AML), chronic myeloid leukemia (CML), acute lymphocytic leukemia (ALL), chronic lymphocytic leukemia (CLL) and secondary leukemia) as well as cancers of the breast, lung, colon, prostate, bladder, and kidney lead to over six million deaths per year (see, e.g., Wang et al., Oncogene 19:1519-1528 (2000)). Standard approaches to cure cancer have centered around a combination of surgery, radiation and chemotherapy. These approaches have resulted in some dramatic successes in certain malignancies. However, cancer is often incurable, when diagnosed beyond a certain stage. Alternative approaches to therapy are needed.

Proteolytic degradation of the extracellular matrix plays a crucial role both in cancer invasion and non-neoplastic tissue remoldeling, and in both cases it is accomplished by a number of proteases. Best known are the plasminogen activation system that leads to the formation of the serine protease plasmin, and a number of matrix metalloproteinase, including collagenases, gelatinases and stromelysins (Dano, K., et al., APMIS, 107:120-127 (1999)).

Among the MMPs, MMP-2 (gelatinase A), MMP-9 (gelatinase B) and membrane-type 1 MMP (MT1-MMP) are reported to be most related to invasion and metastasis in various human cancers (Crawford, H. C., et al., Invasion Metastasis, 14:234-245 (1995); Garbisa, S., et al., Cancer Res., 47:1523-1528 (1987); Himelstein, B. P., et al., Invest. Methods, 14:246-258 (1995); Juarez, J., et al., Int. J. Cancer, 55:10-18 (1993); Kohn, E. C., et al., Cancer Res., 55:1856-1862 (1995); Levy, A. T., et al., Cancer Res., 51:439-444 (1991); Mignatti, P., et al., Physiol. Rev., 73:161-195 (1993); Montgomery, A. M., et al., Cancer Res., 53:693-700 (1993); Stetler-Stevenson, W. G., et al., Annu Rev Cell Biol, 9:541-573 (1993); Stetler-Stevenson, W. G., Invest. Methods, 14:4664-4671 (1995); Davidson, B., et al., Gynecol. Oncol., 73:372-382 (1999); Webber, M. M., et al., Carcinogenesis, 20:1185-1192 (1999); Johansson, N., et al., Am J Pathol, 154:469-480 (1999); Ries, C., et al., Clin Cancer Res., 5:1115-1124 (1999); Zeng, Z. S., et al., Carcinogenesis, 20:749-755 (1999); Gokaslan, Z. L., et al., Clin Exp Metastasis, 16:721-728 (1998); Forsyth, P. A., et al., Br J Cancer, 79:1828-1835 (1999); Ozdemir, E., et al., J Urol, 161:1359-1363 (1999); Nomura, H., et al., Cancer. Res., 55:3263-3266 (1995); Okada, Y., et al., Proc. Natl. Acad. Sci. USA., 92:2730-2734 (1995); Sato, H., et al., Nature, 370:61-65 (1994); Chen, W. T., et al., Ann NY Acad Sci, 878:361-371 (1999); Sato, T., et al., Br J Cancer, 80:1137-43 (1999); Polette, M., et al., Int J Biochem cell Biol., 30:1195-1202 (1998); Kitagawa, Y., et al., J Urol., 160:1540-1545; Nakada, M., et al., Am J Pathol., 154:417-428 (1999); Sato, H., et al., Thromb Haemost, 78:497-500 (1997)). The important role of MMPs during tumor invasion and metastasis is to break down tissue extracellular matrix and dissolution of epithelial and endothelial basement membranes, enabling tumor cells to invade through stroma and blood vessel walls at primary and secondary sites. MMPs also participate in tumor neoangiogenesis and are selectively upregulated in proliferating endothelial cells in tumor tissues (Schnaper, H. W, et al., J Cell Physiol, 156:235-246 (1993); Brooks, P. C., et al., Cell, 92:391-400 (1998); Chambers, A. F., et al., J Natl Cancer Inst, 89:1260-1270 (1997)). Furthermore, these proteases can contribute to the sustained growth of established tumor foci by the ectodomain cleavage of membrane-bound pro-forms of growth factors, releasing peptides that are mitogens for tumor cells and/or tumor vascular endothelial cells (Arribas, J., et al., J Biol Chem, 271:11376-11382 (1996); Suzuki, M., et al., J Biol Chem, 272:31730-31737 (1997)).

MMPs and plasminogen activators (e.g., t-PAR, u-PAR, u-PA, t-PA) are families of enzymes that play a leading role in both the normal turnover and pathological destruction of the extracellular matrix, including tissue remodeling (Birkedal-Hansen, H., Curr Opin Cell Biol, 7:728-735 (1995); Alexander, C. M., et al., Development, 122:1723-1736 (1996)), angiogenesis (Schnaper, H. W, et al., J Cell Physiol, 156:235-246 (1993); Brooks, P. C., et al., Cell, 92:391-400 (1998)), tumor invasion and metastasis formation. The members of the MMP family are multidomain, zinc-containing, neutral endopeptidases and include the collagenases, stromelysins, gelatinases, and membrane-type metalloproteinases (Birkedal-Hansen, H., Curr Opin Cell Biol, 7:728-735 (1995)). It has been well documented in recent years that MMPs and proteins of the plasminogen activation system, e.g., plasminogen activator receptors and plasminogen activators, are overexpressed in a variety of tumor tissues and tumor cell lines and are highly correlated to the tumor invasion and metastasis (Crawford, H. C., et al., Invasion Metastasis, 14:234-245 (1995); Garbisa, S., et al., Cancer Res., 47:1523-1528 (1987); Himelstein, B. P., et al., Invest. Methods, 14:246-258 (1995); Juarez, J., et al., Int. J. Cancer, 55:10-18 (1993); Kohn, E. C., et al., Cancer Res., 55:1856-1862 (1995); Levy, A. T., et al., Cancer Res., 51:439-444 (1991); Mignatti, P., et al., Physiol. Rev., 73:161-195 (1993); Montgomery, A. M., et al., Cancer Res., 53:693-700 (1993); Stetler-Stevenson, W. G., et al., Annu Rev Cell Biol, 9:541-573 (1993); Stetler-Stevenson, W. G., Invest. Methods, 14:4664-4671 (1995); Davidson, B., et al., Gynecol. Oncol., 73:372-382 (1999); Webber, M. M., et al., Carcinogenesis, 20:1185-1192 (1999); Johansson, N., et al., Am J Pathol, 154:469-480 (1999); Ries, C., et al., Clin Cancer Res., 5:1115-1124 (1999); Zeng, Z. S., et al., Carcinogenesis, 20:749-755 (1999); Gokaslan, Z. L., et al., Clin Exp Metastasis, 16:721-728 (1998); Forsyth, P. A., et al., Br J Cancer, 79:1828-1835 (1999); Ozdemir, E., et al., J Urol, 161:1359-1363 (1999); Nomura, H., et al., Cancer. Res., 55:3263-3266 (1995); Okada, Y., et al., Proc. Natl. Acad. Sci. USA., 92:2730-2734 (1995); Sato, H., et al., Nature, 370:61-65 (1994); Chen, W. T., et al., Ann NY Acad Sci, 878:361-371 (1999); Sato, T., et al., Br J Cancer, 80:1137-43 (1999); Polette, M., et al., Int J Biochem cell Biol., 30:1195-1202 (1998); Kitagawa, Y., et al., J Urol., 160:1540-1545; Nakada, M., et al., Am J Pathol., 154:417-428 (1999); Sato, H., et al., Thromb Haemost, 78:497-500 (1997)).

However, catalytic manifestations of MMP and plasminogen activators are highly regulated. For example, the MMPs are expressed as inactive zymogen forms and require activation before they can exert their proteolytic activities. The activation of MMP zymogens involves sequential proteolysis of N-terminal propeptide blocking the active site cleft, mediated by proteolytic mechanisms, often leading to an autoproteolytic event (Springman, E. B., et al., Proc Natl Acad Sci USA, 87:364-368 (1990); Murphy, G., et al., APMIS, 107:38-44 (1999)). Second, a family of proteins, the tissue inhibitors of metalloproteinases (TIMPs), are correspondingly widespread in tissue distribution and function as highly effective MMP inhibitors (Ki˜10⁻¹⁰ M) (Birkedal-Hansen, H., et al., Crit Rev Oral Biol Med, 4:197-250 (1993)). Though the activities of MMPs are tightly controlled, invading tumor cells that utilize the MMP's degradative capacity somehow circumvent these negative regulatory controls, but the mechanisms are not well understood.

With respect to the plasminogen activation system, two plasminogen activators are known, the urokinase-type plasminogen activator (uPA) and the tissue-type plasminogen activator (tPA), of which uPA is the one primarily involved in extracellular matrix degradation (Dano, K., et al., APMIS, 107:120-127 (1999)). uPA is a 52 kDa serine protease which is secreted as an inactive single chain proenzyme (pro-uPA) (Nielsen, L. S., et al., Biochemistry, 21:6410-6415 (1982); Petersen, L. C., et al., J. Biol. Chem., 263:11189-11195 (1988)). The binding domain of pro-uPA is the epidermal growth factor-like amino-terminal fragment (ATF; aa 1-135, 15 kDa) that binds with high affinity (Kd=0.5 mM) to urokinase-type plasminogen activator receptor (uPAR) (Cubellis, M. V., et al., Proc. Natl. Acad. Sci. U.S.A., 86:4828-4832 (1989)), a GPI-linked receptor. uPAR is a 60 kDa three domain glycoprotein whose N-terminal domain 1 contains the high affinity binding site for ATF of pro-uPA (Ploug, M., et al., J. Biol. Chem., 266:1926-1933 (1991); Behrendt, N., et al., J. Biol. Chem., 266:7842-7847 (1991)). uPAR is overexpressed on a variety of tumors, including monocytic and myelogenous leukemias (Lanza, F., et al., Br. J. Haematol., 103:110-123 (1998); Plesner, T., et al., Am. J. Clin. Pathol., 102:835-841 (1994)), and cancers of the breast (Carriero, M. V., et al., Clin. Cancer Res., 3:1299-1308 (1997)), bladder (Hudson, M. A., et al., J. Natl Cancer Inst., 89:709-717 (1997)), thyroid (Ragno, P., et al., Cancer Res., 58:1315-1319 (1998)), liver (De Petro, G., et al., Cancer Res., 58:2234-2239 (1998)), pleura (Shetty, S., et al., Arch. Biochem. Biophys., 356:265-279 (1998)), lung (Morita, S., et al., Int. J. Cancer, 78:286-292 (1998)), pancreas (Taniguchi, T., et al., Cancer Res., 58:4461-4467 (1998)), and ovaries (Sier, C. F., et al., Cancer Res., 58:1843-1849 (1998)). Pro-uPA binds to uPAR by ATF, while the binding process does not block the catalytic, carboxyl-terminal domain. By association with uPAR, pro-uPA gets near to and subsequently activated by trace amounts of plasmin bound to the plasma membrane by cleavage of the single chain pro-uPA within an intra-molecular loop held closed by a disulfide bridge. Thus the active uPA consists of two chains (A+B) held together by this disulfide bond (Ellis, V., et al., J. Biol. Chem., 264:2185-2188 (1989)).

Plasminogen is present at high concentration (1.5-2.0 μM) in plasma and interstitial fluids (Dano, K., et al., Adv. Cancer Res., 44:139-266 (1985)). Low affinity, high capacity binding of plasminogen to cell-surface proteins through the lysine binding sites of plasminogen kringles enhances considerably the rate of plasminogen activation by uPA (Ellis, V., et al., J. Biol. Chem., 264: 2185-2188 (1989); Stephens, R. W., et al., J. Cell Biol., 108:1987-1995 (1989)). Active uPA has high specificity for Arg560-Val561 bond in plasminogen, and cleavage between these residues gives rise to more plasmin that is referred to as “reciprocal zymogen activation” (Petersen, L. C., Eur. J. Biochem., 245:316-323 (1997)). The result of this system is efficient generation of active uPA and plasmin on cell surface. In this context, uPAR serves as a template for binding and localization of pro-uPA near to its substrate plasminogen on plasma membrane.

Unlike uPA, plasmin is a relatively non-specific protease, cleaving many glycoproteins and proteoglycans of the extracellular matrix, as well as fibrin (Liotta, L. A., et al., Cancer Res., 41:4629-4636 (1981)). Therefore, cell surface bound plasmin mediates the non-specific matrix proteolysis which facilitates invasion and metastasis of tumor cells through restraining tissue structures. In addition, plasmin can activate some of the matrix metalloproteases which also degrade tissue matrix (Werb, Z., et al., N. Engl. J. Med., 296:1017-1023 (1977); DeClerck, Y. A., et al., Enzyme Protein, 49:72-84 (1996)). Plasmin can also activate growth factors, such as TGF-β, which may further modulate stromal interactions in the expression of enzymes and tumor neo-angiogenesis (Lyons, R. M., et al., J. Cell Biol., 106:1659-1665 (1988)). Plasminogen activation by uPA is regulated by two physiological inhibitors, plasminogen activator inhibitor-1 and 2 (PAI-1 and PAI-2) (Cubellis, M. V., et al., Proc. Natl. Acad. Sci. U.S.A., 86:4828-4832 (1989); Ellis, V., et al., J. Biol. Chem., 265:9904-9908 (1990); Baker, M. S., et al., Cancer Res., 50:4676-4684 (1990)), by formation 1:1 complex with uPA. Plasmin generated in the cell surface plasminogen activation system is relatively protected from its principle physiological inhibitor α2-antiplasmin (Ellis, V., et al., J. Biol. Chem., 266:12752-12758 (1991)).

uPA and tPA possess an extremely high degree of structure similarity (Lamba, D., et al., J. Mol. Biol., 258:117-135 (1996); Spraggon, G., et al., Structure, 3:681-691 (1995)), share the same primary physiological substrate (plasminogen) and inhibitors (PAI-1 and PAI-2) (Collen, D., et al., Blood, 78:3114-3124 (1991)), and exhibit restricted substrate specificity. By using substrate phage display and substrate subtraction phage display approaches, recent investigations had identified substrates that discriminate between uPA and tPA, showing the consensus substrate sequences with high selectivity by uPA or tPA (Ke, S. H., et al., J. Biol. Chem., 272:20456-20462 (1997); Ke, S. H., et al., J. Biol. Chem., 272:16603-16609 (1997)).

The close association between MMP and plasminogen activator overexpression and tumor metastasis has been noticed for a decade. For example, the contributions of MMPs in tumor development and metastatic process lead to the development of novel therapies using synthetic inhibitors of MMPs (Brown, P. D., Adv Enzyme Regul, 35:293-301 (1995); Wojtowicz-Praga, S., et al., J Clin Oncol, 16:2150-2156 (1998); Drummond, A. H., et al., Ann NY Acad Sci, 30:228-235 (1999)). Among a multitude of synthetic inhibitors generated, Marimastat is already clinically employed in cancer treatment (Drummond, A. H., et al., Ann NY Acad Sci, 30:228-235 (1999)). However, these inhibitors only slow growth and do not eradicate the tumors. Mutant anthrax protective antigen fusion protein (PA) molecules in which the furin cleavage site is replaced by an MMP or plasminogen activator target site have been used to deliver compounds such as toxins to the cell, thereby killing the cell (see, e.g., Liu et al., J. Biol. Chem. 276:17976-17984 (2001); Liu et al., Cancer Res. 60:6061-6067 (2000); and WO 01/21656).

In addition to overexpression of MMP and PA receptors, some cancer cells including acute myelogenous leukemia cells, overexpress receptors for cytokines (e.g., GM-CSF, IL-3) and growth factors (e.g., EGF) (see, e.g., Testa et al., Blood 100(8):2980-8 (2002); Hall et al., Toxicol. Appl. Pharmacol. 50(1):91-7 (1998); and Vinante et al., Blood 93(5):1715-23 (1999)). It has also been shown that treatment with cytokines (e.g., IL-2) can be effective against acute myelogenous leukemia (see, e.g., Toren et al., Med. Oncol. 12(3):177-86 (1995)). Thus, cytokines and growth factors which have receptors overexpressed on the surface of cells (e.g., cancer cells, virally infected cells, bacterially infected cells, and cells that play a role in autoimmune disorders) could potentially be used to target therapeutic or diagnostic compounds to such cells.

Diphtheria toxin fusion proteins comprising residues 1-388 of Diphtheria toxin fused to GM-CSF or IL-3 have also been described (see, e.g., Hall et al., Leukemia. 13:629-633 (1999); Hotchkiss et al., Toxicol. Appl. Pharmacol. 158:152-160 (1999); and Black et al. Leukemia 17(1):155-9 (2003)). However, patients treated with the Diphtheria toxin-GMCSF fusion protein experienced acute liver toxicity, including fatal liver failure within week after treatment (see, e.g., Frankel et al., Clin. Cancer Res. 8:1004-1013 (2002) and Hall et al., Clin. Immunol. 100(2): 191-197 (2001)).

Thus, there is a need in the art for compositions and methods for selectively targeting and/or killing cells that overexpress cytokine receptors and growth factor receptors on their surface (e.g., cancer cells, virally infected cells, bacterially infected cells, and cells that play a role in autoimmune disorders). The present invention fulfills this and other needs.

### BRIEF SUMMARY OF THE INVENTION

The present invention provides compositions and methods for selectively targeting and/or killing cells that overexpress cytokine receptors and growth factor receptors on their surface (e.g., cancer cells, virally infected cells, bacterially infected cells, and cells that play a role in autoimmune disorders). In particular, the invention provides mutant Diphtheria toxin (DT) fusion proteins (i.e., DT fusion proteins), in which the native DT furin cleavage site is replaced by sequences specifically cleaved by MMPs or plasminogen activators and the DT is fused to a heterologous polypeptide, e.g., a cytokine or growth factor. The compounds can be diagnostic or therapeutic agents. Preferably the compounds are delivered to the cells of a human subject suffering from cancer, thereby killing the cancer cells and treating the cancer.

One embodiment of the invention provides nucleic acids encoding Diphtheria toxin fusion proteins comprising (1) residues 1-388 of Diphtheria toxin, wherein the native furin cleavage site has been substituted for a cleavage site for matrix metalloproteinase or a plasminogen activator and (2) a heterologous polypeptide that binds to a protein expressed on the surface of the cell. In some embodiments, the matrix metalloproteinase is MMP-2 (gelatinase A), MMP-9 (gelatinase B) or membrane-type 1 MMP (MT1-MMP). In some embodiments, the tissue plasminogen activator is tPA (tissue-type plasminogen activator) or u-PA (urokinase-type plasminogen activator). In some embodiments, the heterologous polypeptide is a cytokine (e.g., IL-2, GM-CSF, IL-4, IL-5, IL-6, IL-10, or IL-12) or a growth factor (e.g., epidermal growth factor (EGF), transforming growth factor (TGF), or fibroblast growth factor (FGF)). In some embodiments, the nucleic acid encodes a Diphtheria toxin fusion protein comprising (1) residues 1-388 of Diphtheria toxin, wherein the native furin cleavage site has been substituted for a cleavage site for uorkinase plasminogen activator and (2) GM-CSF. In some embodiments, the nucleic acids comprise the nucleotide sequences set forth in any one of SEQ ID NOS: 2-12. In addition, the invention provides vectors comprising the nucleic acids described above, and host cells comprising such vectors. The invention also provides proteins and polypeptides encoded by the nucleic acids described above. The invention further provides pharmaceutical compositions comprising polypeptides encoded by the nucleic acids described above and a pharmaceutically acceptable carrier.

Another embodiment of the invention provides a method of treating cancer by administering to a subject a Diphtheria toxin fusion protein comprising (1) residues 1-388 of Diphtheria toxin, wherein the native furin cleavage site has been substituted for a cleavage site for matrix metalloproteinase or a plasminogen activator and (2) a heterologous polypeptide that binds to a protein expressed on the surface of the cell. In some embodiments, the matrix metalloproteinase is MMP-2, MMP-9, or MMP1-MMP. In some embodiments, the tissue plasminogen activator is tPA or uPA. In some embodiments, the matrix metalloproteinase-recognized cleavage site is GPLGMLSQ or GPLGLWAQ. In some embodiments, the heterologous polypeptide is a cytokine (e.g., IL-2, GM-CSF, IL-4, IL-5, IL-6, IL-10, or IL-12) or a growth factor (e.g., EGF, hGF, or FGF). In another embodiment, the plasminogen activator-recognized cleavage site is GSGRSA, GSGKSA, or QRGRSA. In some embodiments, the Diphtheria toxin fusion protein comprises (1) residues 1-388 of Diphtheria toxin, wherein the native furin cleavage site has been substituted for a cleavage site for uorkinase plasminogen activator and (2) GM-CSF. In some embodiments, the DT fusion protein is encoded by the nucleotide sequence set forth in any one of SEQ ID NOS: 2-13. In some embodiments, the cancer is acute myelogenous leukemia, monocytic leukemia, lung cancer, breast cancer, bladder cancer, thyroid cancer, liver cancer, lung cancer, pleural cancer, pancreatic cancer, ovarian cancer, cervical cancer, colon cancer, fibrosarcoma, neuroblastoma, glioma, or melanoma.

A further embodiment of the invention provides a method of targeting a compound to a cell overexpressing a cytokine receptor or a growth factor receptor by administering to the cell a Diphtheria toxin fusion protein comprising Diphtheria toxin fusion protein comprising (1) residues 1-388 of Diphtheria toxin, wherein the native furin cleavage site has been substituted for a cleavage site for matrix metalloproteinase or a plasminogen activator and (2) a heterologous polypeptide that binds to a protein expressed on the surface of the cell. In some embodiments, the matrix metalloproteinase is MMP-2, MMP-9, or MMP1-MMP. In some embodiments, the tissue plasminogen activator is tPA or uPA. In some embodiments, the matrix metalloproteinase-recognized cleavage site is GPLGMLSQ or GPLGLWAQ. In some embodiments, the heterologous polypeptide is a cytokine (e.g., IL-2, GM-CSF, IL4, IL-5, IL-6, IL-10, or IL-12) or a growth factor (e.g. EGF, hGF, or FGF). In another embodiment, the plasminogen activator-recognized cleavage site is GSGRSA, GSGKSA, or QRGRSA. In some embodiments, the cell also overexpresses a matrix metalloproteinase or a plasminogen activator. In some embodiments, the Diphtheria toxin fusion protein comprises (1) residues 1-388 of Diphtheria toxin, wherein the native furin cleavage site has been substituted for a cleavage site for uorkinase plasminogen activator and (2) GM-CSF.

### BRIEF DESCRIPTION OF THE DRAWINGS AND TABLES

FIG. 1 depicts a schematic illustration uptake of DTGM by cells expressing a GM-CSF receptor. DTGM binds to the GM-CSF receptor on the cell surface and is taken up by receptor mediated endocytosis. DTGM is cleaved furin or a furin-like protease along the endocytic pathway. In the acidic environment of the endosome, the A fragment of DTGM translocates into the cytosol, leading to ADP ribosylation of EF2, inhibition of protein synthesis, and cell death.

FIG. 2 depicts a schematic illustration of mutant DT fusion proteins that can be specifically processed by MMPs. Human GM-CSF was recombinantly fused to the C-terminus of modified DT388. The table represents the sequence modified in the furin sensitive surface loop of DTGM that generate cleavage sites recognized by furin, uPA, or MMP as indicated. To generate DTGM-U2, the native furin cleavage site was replaced by GSGRSA, a urokinase plasminogen cleavage site. To generate DTGM-U3, the native furin cleavage site was replaced by GSGKSA, a urokinase plasminogen cleavage site. To generate DTGM-L1, the native furin cleavage site was replaced by GPLGMLSQ, a matrix metalloproteinase cleavage site.

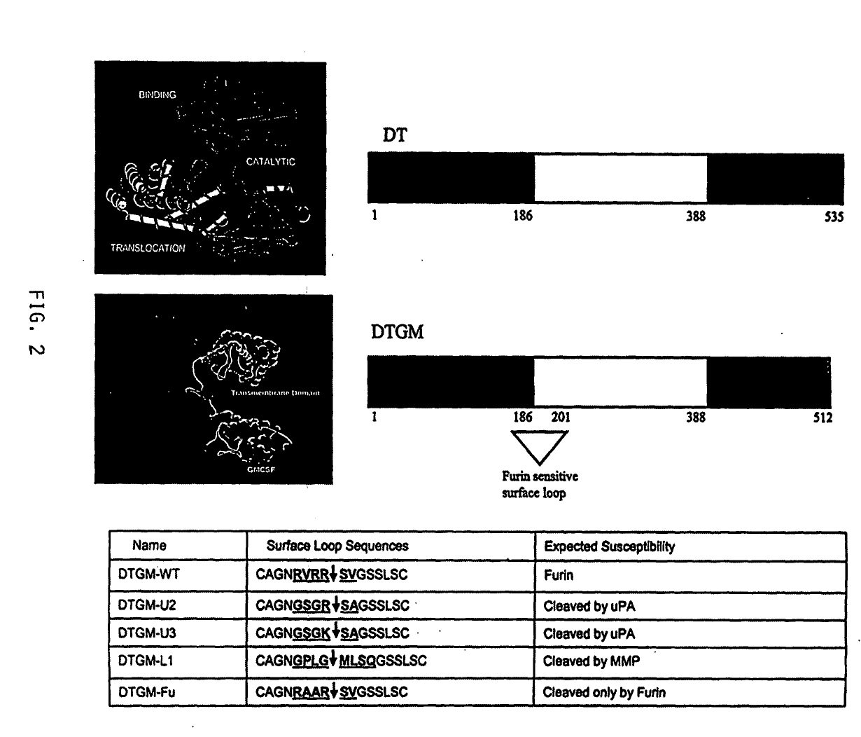

FIG. 3 depicts a schematic illustration of production of mutant DT-GMCSF fusion proteins. pRKDTGM encoding the modified diphtheria toxin GM-CSF fusion protein was transformed into E. coli (BL2) cells which were then incubated at 37° C. in Superbroth, and induced with 1 mM IPTG for 3 hours. Cells were lysed, inclusion bodies were isolated, washed with TES buffer with Triton X-100, and denatured in guanidine-HCl with DTT. Soluble proteins were refolded for 48 hours in buffer containing L-arginine and glutathione. The isolated protein was then dialyzed, filter sterilize, and purified over columns.

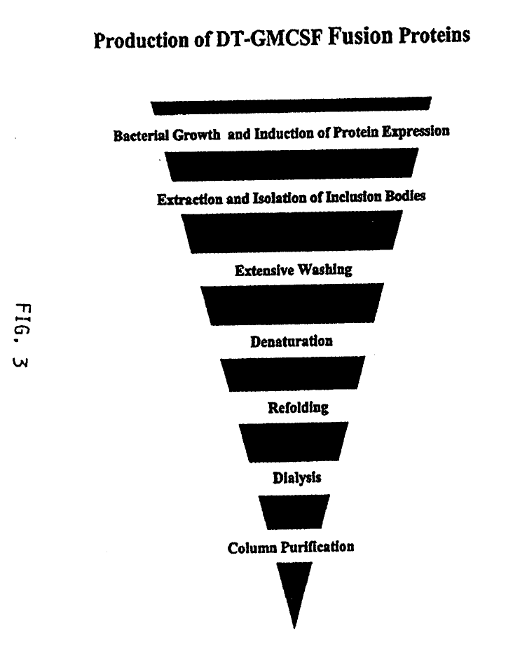

FIG. 4 illustrates data showing cytotoxicity of mutant DTGM fusion proteins to U397 human leukemia cells, a GM-CSF receptor positive cell line. U397 cells were plated in a 96 well plate at 4×10⁴ cells per well per day prior to treatment with mutant DTGM fusion proteins. Cells were incubated in the presence or absence of pro-uPA for one hour before the addition of mutant DTGM fusion proteins. All cells were incubated with serial dilutions of DTGM, DTGM-U2, DTGM-U3 or DTGM-Fu fusion proteins for 48 hours at 37° C. MTS/PMS was added to determined cell viability at 48 hours.

FIG. 5 illustrates data showing cytotoxicity of mutant DTGM fusion proteins to CEM-SS cells, a GM-CSF receptor negative cell line. Cells treated with DTGM-U2 and DTGM-U3 were preincubated with pro-uPA (100 ng/ml) for one hour before addition of DTGM proteins. All cells were incubated with serial dilutions of DT, DTGM, DTGM-U2, DTGM-U3, DTGM-L1, and DTGM-Fu fusion proteins for 48 hours at 37° C. MTS/PMS was added to determined cell viability at 48 hours.

FIG. 6 illustrates data showing pro-uPA cytotoxicity of DTGM-U2 to human leukemia U937 cells. Human leukemia U937 cells were treated with multiple concentrations of DTGM-Us and pro-uPA for 48 hours. Then MTT was added to determine cell viability. Live cells converted MTT to blue dye, which precipitated in cytosol, while dead cells remained colorless. The cytotoxicity of DTGM-U2 to U937 cells was dependent on the presence of pro-uPA.

FIG. 7 illustrates data showing that DTGM-U2 is toxic to human leukemia TF1-vRAF cells. Cells were treated with various concentrations of DTGM alone or DTGM-U2 with and without exogenous human pro-uPA (50 ng/ml) for 48 hours. Thymidine incorporation assays were then performed to determine the cytotoxicity of the mutant DT fusion proteins to the cells. Killing was observed even without exogenous pro-uPA. In the presence of pro-uPA, DTGM-U2 was even more cytotoxic to the leukemia cells.

FIG. 8 illustrates data from proliferation inhibition assays on cell lines treated with DTGM-U2. Proliferation inhibition assays on three AML cell lines: TF1-vRaf (FIG. 8A), ML1 (FIG. 8B) and Monomac 6 (FIG. 8C) using DTGM (triangle), DTGM-U2 (inversed triangle) and DTGM-U2+exogenous pro-uPA (square). The X axis represents the Log of the molar drug concentration and the Y axis represents cell viability expressed as percent control of ³H-thymidine incorporation in counts per minute (CPM). TF1-vRaf was sensitive to DTGM-U2 (IC₅₀=3.14 pM) and the sensitivity was enhanced by the addition of exogenous pro-uPA (IC₅₀=0.26 pM) and became similar to that of DTGM (IC₅₀=0.64 pM) (A). ML-1 was not sensitive to DTGM-U2 unless exogenous pro-uPA was added (IC₅₀=30 pM). IC₅₀ for DTGM was 22 pM (FIG. 8B). Monomac 6 was not sensitive to DTGM-U2 even when exogenous pro-uPA was added (FIG. 8C). FIG. 8D shows data from a blocking assay and proliferation inhibition assay on TF1-vRaf with DTGM-U2 (triangle), DTGM-U2+anti-uPA (square) and DTGM-U2+anti-GMCSF (inversed triangle). On the X axis is the log molar drug concentration, on the Y axis is the percentage of control ³H-thymidine incorporation. Both anti-GMCSF and anti-uPA greatly decreased DTGM-U2 efficacy (IC₅₀=400 pM and 0.67 μM respectively) compared to an IC₅₀=2.3 pM for DTGM-U2 alone, thus demonstrating the dual specificity of DTGM-U2 which requires the expression of both GMCSFR and the uPA/uPAR protease system.

FIG. 9 illustrates data showing uPA receptor levels and GM-CSFR levels in cells used in cytotoxicity assays. FIG. 9A shows uPAR levels as determined by ¹²⁵I-ATF receptor binding assay. FIG. 9B shows HL60 GMCSFR levels as determined by ¹²⁵I-GMCSF receptor binding assay. uPAR expression levels of Sig M5. The X axis represents the concentration of ¹²⁵I-ATF in pg/ml, the Y axis represents the amount of cell bound ¹²⁵I-ATF in CPM. FIG. 9C shows uPAR levels expressed as number of receptors/cell in the 13 AML cell lines. DTGM-U2 resistant cells (white) have lower uPAR expression levels than DTGM-U2 sensitive cells (light gray: without the addition of pro-uPA, dark gray: requiring the addition of pro-uPA).

FIG. 10 illustrates data showing the uPA levels and plasminogen activator inhibitor 1 (PAI-1) levels in cells insensitive to DTGM-U2, cells sensitive to DTGM-U2 alone, and cells sensitive to DTGM-U2 in the presence of exogenous pro-uPA. FIG. 10A depicts the average uPA levels (ng/ml) in the supernatant of each of the three categories of AML cell lines as determined by ELISA. Non-sensitive cell lines (white) had the highest concentrations of total uPA in their supernatants which indicates that DTGM-U2 efficacy is determined by uPAR expression levels rather than uPA concentration. Cell lines that were sensitive to DTGM-U2 only when exogenous pro-uPA was added (dark gray) had significantly lower total uPA levels in their supernatants as compared to cells that do not require the addition of exogenous pro-uPA (light gray) (p=0.04). FIG. 10 B depicts the total PAI-1 concentrations in the supernatants of the AML cell lines as determined by ELISA. PAI-1 levels did not correlate with DTGM-U2 efficacy.

FIG. 11 is Table 1 which summarizes data showing the sensitivity of AML cells lines treated with: (1) DTGM-U2; (2) DTGM; or (3) and DTGM-U2 and exogenous pro-uPA.

FIG. 12 is Table 2 which summarizes data comparing the efficacy of DTGM with GM-CSFR expression levels.

FIG. 13 is Table 3 which summarizes data comparing uPAR expression levels, uPA levels, and DTGM-U2 efficacy on cell lines sensitive to DTGM.

FIG. 14 is Table 4 which summarizes data showing the cytotoxicity of DTGM, DTGM-U2, and DTAT on normal cells.

### DETAILED DESCRIPTION OF THE INVENTION

### I. Introduction

The present invention provides compositions and methods for diagnosing and treating cancer. In particular, the invention provides modified Diphtheria toxin (DT) fusion proteins targeted to a heterologous polypeptide overexpressed on the surface of a cell (e.g., a cell surface receptor), and which are specifically activated by MMPs or plasminogen activators to specially kill MMP- or and plasminogen activators-expressing tumor cells and methods of using such modified DT fusion proteins. DT fusion proteins comprise (1) a residues 1-388 of DT wherein the native furin recognition site has been replaced by sequences susceptible to cleavage by MMPs or and plasminogen activators and (2) a heterologous protein, typically a cytokine or growth factor (e.g., GM-CSF, IL-2, or EGF). For example, DTGM-L1 and DTGM-L2 are DT-GMCSF fusion proteins in which the native furin recognition site has been replaced by sequences susceptible to cleavage by MMP's and DTGM-U2 and DTGM-U3 are DT-GMCSF fusion proteins in which the native furin recognition site has been replaced by sequences susceptible to cleavage by urokinase plasminogen activators. DTEGF-L1 and DTEGF-L2 are DT-EGF fusion proteins in which the native furin recognition site has been replaced by sequences susceptible to cleavage by MMP's and DTEGF-U2 and DTEGF-U3 are DT-EGF fusion proteins in which the native furin recognition site has been replaced by sequences susceptible to cleavage by urokinase plasminogen activators. DTIL2-L1 and DTIL2-L2 are DT-L2 fusion proteins in which the native furin recognition site has been replaced by sequences susceptible to cleavage by MMP's and DTIL2-U2 and DTIL2-U3 are DT-IL2 fusion proteins in which the native furin recognition site has been replaced by sequences susceptible to cleavage by urokinase plasminogen activators.

In some embodiments, the modified DT fusion proteins are specifically targeted to cells overexpressing a receptor for the heterologous protein (e.g. a growth factor or cytokine). In some embodiments, the modified DT fusion proteins are specifically targeted to cancer cells overexpressing a receptor for the heterologous protein (e.g. a growth factor or cytokine), thereby specifically delivering the Diphtheria toxin to such cells. In some embodiments, the modified DT fusion proteins are specifically targeted to cells overepressing both a plasminogen activator (e.g., uPA) and a receptor for the heterologous protein (e.g., a growth factor or cytokine such as GM-CSF). In other embodiments, the cells, are virally infected cells, bacterially infected cells, or cells that play a role in autoimmune disorders.

These proteins provide a way to specifically kill cancer cells and thus treat cancers including, e.g., leukemias and lymphomas such as acute myeloid leukemia, chronic myeloid leukemia, acute lymphocytic leukemia, chronic lymphocytic leukemia, multiple myeloma, Hodgkin's lymphoma, non-Hodgkin's lymphoma, and secondary leukemia, and other cancers such as melanoma, colon cancer, breast cancer, bladder cancer, thyroid cancer, liver cancer, pleural cancer, lung cancer, prostate cancer, ovarian cancer, pancreatic cancer, head and neck cancer, kidney cancer, stomach cancer, brain cancer) without serious damage to normal cells. This method can also be applied to non-cancerous inflammatory cells or virally infected cells that contain high amounts of cell-surface associated MMPs or plasminogen activators and express receptors for cytokines and growth factors. These mutant DT fusion proteins are thus useful as therapeutic agents to specifically kill cancer cells.

It was originally thought that the role of MMPs and plasminogen activators was simply to break down tissue barriers to promote tumor invasion and metastasis. It is now understood, for example, that MMPs also participate in tumor neoangiogenesis and are selectively upregulated in proliferating endothelial cells. Therefore, these modified bacterial toxins may have the advantageous properties that targeted to not only cancer cells themselves but may also the dividing vascular endothelial cells which essential to neoangiogenesis in tumor tissues. Therefore, the MMP targeted toxins may also kill cancer cells by starving the cells of necessary nutrients and oxygen.

### II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. Molecular Cloning: A Laboratory Manual, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

The terms “Diphtheria toxin” and “DT” refer to a 535 amino acid polypeptide secreted by Corynbacterium diphtheria or a subsequence thereof. DT comprises three domains: (1) an N terminal catalytic domain (aa 1-186); (2) a translocation domain (aa 187-388); and (3) a cell binding domain (aa 389-535) (see, e.g., Frankel et al., Protein Peptide Lett. 9(10:1-14 (2002) and Genbank Accession No. A04646). The catalytic domain and translocation domain are connected by a furin-sensitive disulfide loop. DT binds to a heparin-binding epidermal growth factor-like growth factor expressed on a cell surface via the cell binding domain and associates with CD9 and heparin sulfate proteoglycan. The DT complex is then internalized into the cell (i.e., into endosomes). Once in the endosomal compartment, DT undergoes furin cleavage which releases the catalytic domain which is then translocated through the endosomal membrane and into the cytosol. Once in the cytosol, the catalytic domain ASP-ribosylates elongation factor 2 (EF2), thus blocking protein synthesis and killing the cell.

The terms “mutant,” “modified,” and “derivative” refer to the manipulation, of nucleic acid sequence or amino acid sequence encoding a protein, by recombinant or synthetic methods, resulting in a change in the nucleic acid sequence or amino acid sequence, respectively, such that the sequence is different from the original or unmanipulated sequence. For example, an nucleic acid sequence or amino acid sequence encoding a protein can be manipulated by extending, shortening, replacing, or otherwise changing the original or unmanipulated, by using the recombinant or synthetic methods described herein or known to one of skill in the art.

The terms “mutant diphtheria toxin” or “mutant DT” as used herein refer to a recombinant or synthetic protein that is a modified, mutant, or derivative form of the native DT, that comprises a cleavage site of a specific extracellular protease in place of the furin cleavage site of the native DT and binds to a receptor on the surface of a cell expressing an extracellular protease. The protease cleavage site may be a native protease cleavage site, a mutant, modified, or derivative form of a native protease cleavage site, or an artificial or synthetic cleavage site, that is cleaved by a specific extracellular protease. For example, see PCT application WO 01/21656. In addition, the mutant, modified, derivative, artificial or synthetic protease cleavage site may confer an activity or specificity that differs from the native protease cleavage site, for example, an increase or decrease in protease cleavage activity or specificity. Further, the DT may be constructed as a fusion protein, for example, a comprising an amino acid sequence encoding a DT and a ligand (e.g., HB-EGF), or subsequences thereof.

A “fusion protein” refers to a protein having at least two polypeptides covalently linked, in which one polypeptide comes from one protein sequence or domain and the other polypeptide comes from another protein sequence or domain. The polypeptides can be linked either directly or via a covalent linker, e.g., an amino acid linker, such as a polyglycine linker, or another type of chemical linker, e.g., a carbohydrate linker, a lipid linker, a fatty acid linker, a polyether linker, e.g., PEG, etc. (see, e.g., Hermanson (1996) Bioconjugate techniques). The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. The term “fusion protein” also refers to conservatively modified variants, polymorphic variants, alleles, mutant, subsequences and interspecies homologues of the polypeptides that make up the fusion protein. Fusion proteins may be produced by covalently linking a chain of amino acids from one protein sequence to a chain of amino acids from another protein sequence, e.g., by preparing a recombinant polynucleotide contiguously encoding the fusion protein. Fusion proteins can comprise 2, 3, 4 or more different chains of amino acids from the same or different species. The different chains of amino acids in a fusion protein may be directly spliced together or may be indirectly spliced together via a chemical linking group or an amino acid linking group. The fusion protein may optionally comprise other components, as described in more detail herein.

A “DT fusion protein” comprises a DT polypeptide and a heterologous polypeptide. The heterologoud polypeptide binds to a protein overexpressed on the surface of a cell, e.g., a cancer cell or a virally infected cells. Suitable heterologous polypeptides include, e.g., cytokines or growth factors such as, IL-2 (Genbank Accession No. M22005), GM-CSF (Genbank Accession No. M13207), or EGF (Genbank Accession No. NM_{—}001963).

The phrase “extracellular protease” refers to a protease localized on the surface of a cell, and is not limited to a protease embedded or attached, directly or indirectly, to the cell. An example of a extracellular protease is a protease capable of specifically cleaving a PrAg used in the methods (see, e.g., WO 01/21656). Examples of extracellular proteases are serine, matrix metallo-, and cysteine proteases, but are not limited thereto. Further examples of extracellular proteases are proteases belonging to the following classes of protease: MMP (e.g., MT1-MMP), ADAMS (e.g., ADAM-15), type-I transmembrane serine proteases (e.g., prostasin), type-II transmembrane serin proteases (e.g., matriptase), cathepsins (e.g., cathepsin B), GPI-anchored serine proteases, as described in Frosch et al., APMIS (1999) 107: 28-37; Schlondorff et al., J. Cell Sci. (1999) 112: 3603-3617; Kaushal et al., J. Clinical Investigation (2000) 105: 13351337; Ellerbroek, BioEssays (1999) 21: 940-949; and Hooper et al. (2001) 276: 857-860.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, ÿ-carboxyglutamate, and O-phosphoserine. Amino acid analogs refers to compounds that have the same basic chemical structure as a naturally occurring amino acid, i.e., an y carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Protein”, “polypeptide”, or “peptide” refer to a polymer in which the monomers are amino acids and are joined together through amide bonds, alternatively referred to as a polypeptide. When the amino acids are -amino acids, either the 1-optical isomer or the d-optical isomer can be used. Additionally, unnatural amino acids, for example, -alanine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the d- or l-isomer. The l-isomers are generally preferred. In addition, other peptidomimetics are also useful in the present invention. For a general review, see, Spatola, A. F., in Chemistry and Biochemistry of Amino Acids, Peptides and Proteins, B. Weinstein, eds., Marcel Dekker, New York, p. 267 (1983).

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

The phrase “recombinant protein” as used herein refers to a protein which has been produced by a recombinant cell.

The phrases “enzymatic activity” or “binding activity” or “protease activity” or “cleavage activity” as used herein refer to the activity of a biomolecule, for example a protein, and may be measured by a variety of assays and units as described herein or known to one skilled in the art.

The phrase “catalytic domain” refers to a protein domain, or portion thereof, that is sufficient to catalyze an enzymatic reaction that is normally carried out by the enzyme.

The term “subsequence” refers to a sequence of nucleic acids or amino acids that comprise a part of a longer sequence of nucleic acids or amino acids (e.g., protein) respectively.

The phrase “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

The phrases “recombinant expression cassette” or simply an “expression cassette” refer to a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The phrases “heterologous sequence” or a “heterologous nucleic acid”, as used herein, refers to a sequence that originates from a source foreign to the particular host cell, or, if from the same source, is modified from its native form. Thus, a heterologous gene in a eukaryotic host cell includes the modified form of a native gene that is endogenous to the host cell.

The term “isolated” refers to material that is substantially or essentially free from components which interfere with the activity or use of the material. For cells, nucleic acids, and protein of the invention, the term “isolated” refers to material that is substantially or essentially free from components which normally accompany the material as found in its native state. Typically, isolated proteins or nucleic acids of the invention are at least about 80% pure, usually at least about 90%, and preferably at least about 95% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized.

The phrase “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the sequence encoded by the second nucleic acid sequence.

Samples or assays comprising proteases that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a protease is achieved when the activity value relative to the control is about 80%, preferably 50%, more preferably 25-0%. Activation of a protease is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, antisense molecule, RNAi molecule, etc., to be tested for the capacity to directly or indirectly modulate protease activity. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or amino acid sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or proteins, refers to two or more sequences or subsequences that have at least 60%-70%, preferably 80-85%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g. by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, CURRENT PROTOCOLS IN MOLECULAR BIOLOGY, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, PNAS USA 89:10915 (1989)).

In addition to calculating percent sequence identity, the BLAST algorithm also performs a statistical analysis of the similarity between two sequences (see, e.g., Karlin & Altschul, PNAS USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or proteins are substantially identical is that the protein encoded by the first nucleic acid is immunologically cross reactive with the protein encoded by the second nucleic acid, as described below. Thus, a protein is typically substantially identical to a second protein, for example, where the two peptides differ only by conservative substitutions. Another indication that two nucleic acid sequences are substantially identical is that the two molecules hybridize to each other under stringent conditions, as described below.

The phrase “hybridizing specifically to,” refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 15 C lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and CURRENT PROTOCOLS IN MOLECULAR BIOLOGY, ed. Ausubel, et al. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR PROTOCOLS, A GUIDE TO METHODS AND APPLICATIONS, Academic Press, Inc. N.Y.).

The term “specific” or “specifically” when used with reference to protein binding or protein cleavage, refers to a binding reaction or proteolytic reaction, respectively, which is determinative of the presence of the protein in the presence of a heterogeneous population of proteins and/or other biomolecules. For example, under designated conditions, a specified protein preferentially binds to a particular protein and does not bind in a significant amount to other proteins present in a sample; or under designated conditions, a specified protease preferentially cleaves a particular protein and does not cleave a significant amount of other proteins in a sample. For example, the PrAg proteins used in the methods of the present invention comprise a protease cleavage site that is cleaved by a specific cognate extracellular protease.

“Conservatively modified variations” of a particular nucleic acid sequence refers to those nucleotides of the nucleic acid sequence that encode identical or essentially identical amino acid sequences, or where the nucleic acid sequence does not encode an amino acid sequence, to essentially identical sequences. Because of the degeneracy of the genetic code, a large number of functionally identical nucleic acids encode any given protein. For instance, the codons CGU, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every polynucleotide sequence described herein which encodes a protein also describes every possible silent variation, except where otherwise noted. One of skill will recognize that each codon in a nucleic acid (except AUG, which is ordinarily the only codon for methionine, and UGG which is ordinarily the only codon for tryptophan) can be modified to yield a functionally identical molecule by standard techniques. Accordingly, each “silent variation” of a nucleic acid which encodes a protein is implicit in each described sequence.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art.

One of skill will appreciate that many conservative variations of the fusion proteins and nucleic acid which encode the fusion proteins yield essentially identical products. For example, due to the degeneracy of the genetic code, “silent substitutions” (i.e., substitutions of a nucleic acid sequence which do not result in an alteration in an encoded protein) are an implied feature of every nucleic acid sequence which encodes an amino acid. As described herein, sequences are preferably optimized for expression in a particular host cell used to produce the chimeric endonucleases (e.g., yeast, human, and the like). Similarly, “conservative amino acid substitutions,” in one or a few amino acids in an amino acid sequence are substituted with different amino acids with highly similar properties (see, the definitions section, supra), are also readily identified as being highly similar to a particular amino acid sequence, or to a particular nucleic acid sequence which encodes an amino acid. Such conservatively substituted variations of any particular sequence are a feature of the present invention. See also, Creighton (1984) Proteins, W.H. Freeman and Company. In addition, individual substitutions, deletions or additions which alter, add or delete a single amino acid or a small percentage of amino acids in an encoded sequence are also “conservatively modified variations”.

The practice of this invention can involve the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well known to persons of skill. Examples of these techniques and instructions sufficient to direct persons of skill through many cloning exercises are found in Berger and Kimmel, GUIDE TO MOLECULAR CLONING TECHNIQUES, METHODS IN ENZYMOLOGY volume 152 Academic Press, Inc., San Diego, Calif. (Berger); and CURRENT PROTOCOLS IN MOLECULAR BIOLOGY, F. M. Ausubel et al., eds., Current Protocols, a joint venture between Greene Publishing Associates, Inc. and John Wiley & Sons, Inc., (1999 Supplement) (Ausubel). Suitable host cells for expression of the recombinant polypeptides are known to those of skill in the art, and include, for example, eukaryotic cells including insect, mammalian and fungal cells (e.g., Aspergillus niger)

Examples of protocols sufficient to direct persons of skill through in vitro amplification methods, including the polymerase chain reaction (PCR) the ligase chain reaction (LCR), Qÿ-replicase amplification and other RNA polymerase mediated techniques are found in Berger, Sambrook, and Ausubel, as well as Mullis et al. (1987) U.S. Pat. No. 4,683,202; PCR PROTOCOLS A GUIDE TO METHODS AND APPLICATIONS (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) PNAS USA 86: 1173; Guatelli et al. (1990) PNAS USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in MONOCLONAL ANTIBODIES AND CANCER THERAPY, Alan R. Liss, Inc. (1985); Coligan, CURRENT PROTOCOLS IN IMMUNOLOGY (1991); Harlow & Lane, ANTIBODIES, A LABORATORY MANUAL (1988); and Goding, MONOCLONAL ANTIBODIES: PRINCIPLES AND PRACTICE (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, IMMUNOLOGY (3rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a DT protein, polymorphic variants, alleles, orthologs, and conservatively modified variants, or splice variants, or portions thereof, can be selected to obtain only those polyclonal antibodies that are specifically immunoreactive with DT proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, ANTIBODIES, A LABORATORY MANUAL (1988) for a description of immunoassay formats and conditions that can be used to determine specific immunoreactivity).

The terms “effective amount” or “amount effective to” or “therapeutically effective amount” refers to an amount sufficient to induce a detectable therapeutic response in the subject. The exact dose or amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)). Preferably, the therapeutic response is effective in reducing the proliferation of cancer cells or in inhibiting the growth of cancer cells present in a subject. Assays for determining therapeutic responses are well known in the art.

### III. Diphtheria Toxin Fusion Protein Constructs

In one embodiment, the present invention relates to isolated or purified polynucleotides that encode the Diphtheria toxin fusion proteins. In accordance with the invention, any nucleotide sequence which encodes the amino acid sequence of the fusion protein of interest can be used to generate recombinant molecules which direct the expression of the fusion protein. Using current methods of chemical synthesis, DT proteins can be also be chemically bound to a heterologous polypeptide.

DT fusion proteins can be produced from nucleic acid constructs encoding amino acid residues 1-388 of DT, in which the naturally occurring furin cleavage site has been replaced by an MMP or a plasminogen activator cleavage site, and a hterologous polypeptide (e.g., GM-CSF, IL-2, or EGF). Those of skill in the art will recognize a wide variety of ways to introduce mutations into a nucleic acid encoding DT toxin or to construct a mutant DT toxin-encoding nucleic acid. Such methods are well known in the art (see Sambrook et al., Molecular Cloning, A Laboratory Manual (2^{nd} ed. 1989); Kriegler, Gene Transfer and Expression: A Laboratory Manual (1990); and Current Protocols in Molecular Biology (Ausubel et al., eds., 1994)). In some embodiments, nucleic acids of the invention are generated using PCR. For example, using mutagenic PCR DT fusion protein encoding nucleic acids can be generated by substituting the nucleic acid subsequence that encodes the furin site with a nucleic acid subsequence that encodes a matrix metalloproteinase (MMP) site (e.g., GPLGMLSQ and GPLGLWAQ). Similarly, a mutagenic PCR method can be used to construct the DT fusion proteins in which the furin site is replaced by a plasminogen activator cleavage site (e.g., the uPA and tPA physiological substrate sequence PCPGRVVGG, the uPA favorite sequence GSGRSA, the uPA favorite sequence GSGKSA, or the tPA favorite sequence QRGRSA).

In order to clone full-length coding sequences or homologous variants to generate the DT fusion polynucleotides, labeled DNA probes designed from any portion of the DT nucleotide sequences or their complements may be used to screen a genomic or cDNA library, to identify the coding sequence of each individual component of the fusion protein.

Such clones may be isolated by screening an appropriate expression library for clones that express a full length DT protein. The library preparation and screen may generally be performed using methods known to one of ordinary skill in the art, such as methods described in Sambrook et al., Molecular Cloning: A Laboratory Manual, Cold Spring Harbor Laboratories, Cold Spring Harbor, N.Y. (1989). Briefly, a bacteriophage expression library may be plated and transferred to filters. The filters may then be incubated with a detection reagent. In the context of this invention, a “detection reagent” is any compound capable of binding to the DT protein, which may then be detected by any of a variety of means known to one of ordinary skill in the art. Typical detection reagents contain a “binding agent,” such as Protein A, Protein G, IgG or a lectin, coupled to a reporter group. Preferred reporter groups include enzymes, substrates, cofactors, inhibitors, dyes, radionuclides, luminescent groups, fluorescent groups and biotin. More preferably, the reporter group is horseradish peroxidase, which may be detected by incubation with a substrate such as tetramethylbenzidine or 2,2′-azino-di-3-ethylbenz-thiazoline sulfonic acid. Plaques containing genomic or cDNA sequences that express DT protein are isolated and purified by techniques known to one of ordinary skill in the art. Appropriate methods may be found, for example, in Sambrook et al., supra.

Isolation of coding sequences may also be carried out by the polymerase chain reaction (PCR) using two degenerate oligonucleotide primer pools designed on the basis of the coding sequences disclosed herein. The desired nucleic acids can also be cloned using other well known amplification techniques. Examples of protocols sufficient to direct persons of skill through in vitro amplification methods, including PCR, ligase chain reaction (LCR), Qβ-replicase amplification and other RNA polymerase mediated techniques are found in Sambrook et al., supra, and Ausubel et al. Current Protocols in Molecular Biology (1994), as well as in U.S. Pat. No. 4,683,202; PCR PROTOCOLS A GUIDE TO METHODS AND APPLICATIONS (Innis et al. eds. 1990); Arnheim & Levinson C&EN pp. 36-47 (Oct. 1, 1990); The Journal of NIH Research 3:81-94 (1991); Kwoh et al. (1989) Proc. Natl. Acad. Sci. USA 86:1173; Guatelli et al. (1990) Proc. Natl. Acad. Sci. USA 87:1874; Lomell et al. (1989) J. Clin. Chem. 35:1826; Landegren et al. (1988) Science 241:1077-1080; Van Brunt (1990) Biotechnology 8:291-294; Wu et al. (1989) Gene 4:560; and Barringer et al. (1990) Gene 89:117. Improved methods of cloning in vitro amplified nucleic acids are described in U.S. Pat. No. 5,426,039. Suitable primers for use in the amplification of the nucleic acids of the invention can be designed based on the sequences provided herein.

In accordance with the invention, a polynucleotide of the invention which encodes a fusion protein, fragment thereof, or functional equivalent thereof may be used to generate recombinant nucleic acid molecules that direct the expression of the fusion protein, fragment thereof, or functional equivalent thereof, in appropriate host cells. The fusion polypeptide products encoded by such polynucleotides may be altered by molecular manipulation of the coding sequence.

Due to the inherent degeneracy of the genetic code, other DNA sequences which encode substantially the same or a functionally equivalent amino acid sequence, may be used in the practice of the invention for the expression of the fusion polypeptides. Such DNA sequences include those which are capable of hybridizing to the coding sequences or their complements disclosed herein under low, moderate or high stringency conditions as described herein.

Altered nucleotide sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues, which result in a silent change thus producing a functionally equivalent antigenic epitope. Such conservative amino acid substitutions may be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine, histidine and arginine; amino acids with uncharged polar head groups having similar hydrophilicity values include the following: glycine, asparagine, glutamine, serine, threonine and tyrosine; and amino acids with nonpolar head groups include alanine, valine, isoleucine, leucine, phenylalanine, proline, methionine and tryptophan.

The nucleotide sequences of the invention may be engineered in order to alter the fusion protein coding sequence for a variety of ends, including but not limited to, alterations which modify processing and expression of the gene product. For example, mutations may be introduced using techniques which are well known in the art, e.g., to insert or delete restriction sites, to alter glycosylation patterns, phosphorylation, to create and/or destroy translation, initiation, and/or termination sequences, or to create variations in coding regions, to facilitate further in vitro modification, etc. One of skill will recognize many ways of generating alterations in a given nucleic acid construct. Such well-known methods include, e.g., site-directed mutagenesis, PCR amplification using degenerate oligonucleotides, exposure of cells containing the nucleic acid to chemical mutagenic agents or radiation, chemical synthesis of a desired oligonucleotide (e.g., in conjunction with ligation and/or cloning to generate large nucleic acids) and other well-known techniques (see, e.g., Giliman et al. (1979) Gene 8:81-97; Hutchinson et al. (1978) J. Biol. Chem. 253:6551; Roberts et al. (1987) Nature 328: 731-734). Preferably, the manipulations do not destroy immunogenicity of the fusion polypeptides.

A. Sequence Modifications

Variants of the DT fusion proteins of the invention that retain the ability to inhibit abnormal cell proliferation may be identified by modifying the sequence in one or more of the aspects described above and assaying the resulting fusion protein for the ability to as described in detail below. Naturally occurring variants of the individual polypeptide components of the fusion protein may also be isolated by, for example, screening an appropriate cDNA or genomic library with a DNA sequence encoding each individual polypeptide or a variant thereof.

The above-described sequence modifications may be introduced using standard recombinant techniques or by automated synthesis of the modified fusion protein. For example, mutations can be introduced at particular loci by synthesizing oligonucleotides containing a mutant sequence, flanked by restriction sites enabling ligation to fragments of the native sequence. Following ligation, the resulting reconstructed sequence encodes an analogue having the desired amino acid insertion, substitution, or deletion.

Alternatively, oligonucleotide-directed site-specific mutagenesis procedures can be used to provide a gene in which particular codons are altered according to the substitution, deletion, or insertion required. Exemplary methods of making the alterations set forth above are described by Walder et al. (1986) Gene 42:133; Bauer et al. (1985) Gene 37:73; Craik (1985) BioTechniques January: 12-19; Smith et al. (1981) GENETIC ENGINEERING: PRINCIPLES AND METHODS, Plenum Press; and U.S. Pat. Nos. 4,518,584 and 4,737,462.

Mutations in nucleotide sequences constructed for expression of such DT fusion proteins must, of course, preserve the reading frame of the coding sequences and preferably will not create complementary regions that could hybridize to produce secondary mRNA structures, such as loops or hairpins, which would adversely affect the translation of the mRNA. Although a mutation site may be predetermined, it is not necessary that the nature of the mutation per se be predetermined. For example, in order to select for optimum characteristics of mutants at a given site, random mutagenesis may be conducted at the target codon and the expressed DT fusion protein mutants screened for the desired activity. Not all mutations in a nucleotide sequence which encodes a DT fusion protein will be expressed in the final product. For example, nucleotide substitutions may be made to enhance expression, primarily to avoid secondary structure loops in the transcribed mRNA (see, e.g., European Patent Application 75,444A), or to provide codons that are more readily translated by the selected host, such as the well-known E. coli preference codons for E. coli expression.

B. Expression of DT Fusion Proteins

To obtain high level expression of a nucleic acid (e.g., cDNA, genomic DNA, PCR product, etc. or combinations thereof) encoding a native (e.g., DT or DT fusion protein (e.g., DTGM, DTGM-U2, DTGM, -U3, DTGM-L1, DTGM-L2, DTEGF-L1, DTEGF-L2, DTEGF-U2, DTEGF-U3, DTIL2-L1, DTIL2-L2, DTIL2-U2, and DTIL2-U3DTGM-Fu etc.), one typically subclones the DT fusion protein encoding nucleic acid into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the DT fusion protein encoding nucleic acid are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known in the art and are also commercially available.

In some embodiment, DT fusion protein containing proteins are expressed in non-virulent strains of Bacillus using Bacillus expression plasmids containing nucleic acid sequences encoding the particular DT fusion protein protein (see, e.g., Singh, Y., et al., J Biol Chem, 264:19103-19107 (1989)). The DT fusion protein containing proteins can be isolated from the Bacillus culture using protein purification methods (see, e.g., Varughese, M., et al., Infect Immun, 67:1860-1865 (1999)).

The promoter used to direct expression of a DT fusion protein encoding nucleic acid depends on the particular application. The promoter is preferably positioned about the same distance from the heterologous transcription start site as it is from the transcription start site in its natural setting. As is known in the art, however, some variation in this distance can be accommodated without loss of promoter function. The promoter typically can also include elements that are responsive to transactivation, e.g., Gal4 responsive elements, lac repressor responsive elements, and the like. The promoter can be constitutive or inducible, heterologous or homologous.

In addition to the promoter, the expression vector typically contains a transcription unit or expression cassette that contains all the additional elements required for the expression of the nucleic acid in host cells. A typical expression cassette thus contains a promoter operably linked, e.g., to the nucleic acid sequence encoding the DT fusion protein containing protein, and signals required for efficient expression and termination and processing of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from bacterial proteins, or mammalian proteins such as tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

DNA regions are “operably linked” when they are functionally related to each other. For example, DNA for a signal peptide (secretory leader) is operably linked to DNA for a polypeptide if it is expressed as a precursor which participates in the secretion of the polypeptide; a promoter is “operably linked” to a coding sequence if it controls the transcription of the sequence; or a ribosome binding site is “operably linked” to a coding sequence if it is positioned so as to permit translation. Generally, “operably linked” means contiguous and, in the case of secretory leaders, in reading frame. DNA sequences encoding DT fusion proteins which are to be expressed in a microorganism will preferably contain no introns that could prematurely terminate transcription of DNA into mRNA.

In addition to a promoter sequence, the expression cassette should also contain a transcription termination region downstream of the structural gene to provide for efficient termination and processing, if desired. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors for bacterial use may comprise a selectable marker and a bacterial origin of replication derived from commercially available plasmids comprising genetic elements of the well known cloning vector pBR322 (ATCC 37017). Such commercial vectors include, e.g., pKK223-3 (Pharmacia Fine Chemicals, Uppsala, Sweden), pGEM1 (Promega Biotec, Madison, Wis.), pET28b (Novagen) and pPDM (a modified pET28b, Corixa). These pBR322 “backbone” sections are combined with an appropriate promoter and the structural sequence to be expressed. E. coli is typically transformed using derivatives of pBR322, a plasmid derived from an E. coli species (Bolivar et al. (1977) Gene 2:95). pBR322 contains genes for ampicillin and tetracycline resistance and thus provides simple means for identifying transformed cells. Promoters commonly used in recombinant microbial expression vectors include the β-lactamase (penicillinase) and lactose promoter system (Chang et al. (1978) Nature 275:615; and Goeddel et al. (1979) Nature 281:544), the tryptophan (trp) promoter system (Goeddel et al. (1980) Nucl. Acids Res. 8:4057; and European Patent Application 36,776) and the tac promoter (Maniatis, MOLECULAR CLONING: A LABORATORY MANUAL, COLD SPRING HARBOR LABORATORY, p. 412 (1982)). A particularly useful bacterial expression system uses the phage λ PL promoter and cI857ts thermolabile repressor. Plasmid vectors available from the American Type Culture Collection which incorporate derivatives of the λ PL promoter include plasmid pHUB2, resident in E. coli strain JMB9 (ATCC 37092) and pPLc28, resident in E. coli RR1 (ATCC 53082).

Suitable promoter sequences in yeast vectors include the promoters for alcohol oxidase, metallothionein, 3-phosphoglycerate kinase (Hitzeman et al. (1980) J. Biol. Chem. 255:2073) or other glycolytic enzymes (Hess et al. (1968) J. Adv. Enzyme Reg. 7:149; and Holland et al. (1978) Biochem. 17:4900), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are further described in, e.g., European Patent Application No. 73,657.

Preferred yeast vectors can be assembled using DNA sequences from pBR322 for selection and replication in E. coli (Ampr gene and origin of replication) and yeast DNA sequences including a glucose-repressible ADH2 promoter and α-factor secretion leader. The ADH2 promoter has been described by Russell et al. (1982) J. Biol. Chem., 258:2674 and Beier et al. (1982) Nature 300:724. The yeast α-factor leader, which directs secretion of heterologous proteins, can be inserted between the promoter and the structural gene to be expressed (see, e.g., Kurjan et al. (1982) Cell 30:933; and Bitter et al. (1984) Proc. Natl. Acad. Sci. USA 81:5330. The leader sequence may be modified to contain, near its 3′ end, one or more useful restriction sites to facilitate fusion of the leader sequence to foreign genes. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a DT fusion protein encoding nucleic acid under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of heterologous sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362(Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

After the expression vector is introduced into the cells, the transfected cells are cultured under conditions favoring expression of the DT fusion protein containing protein, which is recovered from the culture using standard techniques identified below.

C. Host Cells

Transformed host cells are cells which have been transformed or transfected with expression vectors constructed using recombinant DNA techniques and which contain sequences encoding DT fusion proteins of the present invention. Transformed host cells may express the desired DT fusion proteins, but host cells transformed for purposes of cloning or amplifying DT DNA do not need to express the DT fusion proteins. Expressed DT fusion proteins will preferably be secreted into the culture medium or supernatant, depending on the DNA selected. One skilled in the art will appreciate that if DT fusion proteins are secreted into the culture supernatant, then they are also soluble in the culture supernatant.

Any of the well known procedures for introducing foreign nucleotide sequences into host cells may be used to introduce the expression vector. These include the use of reagents such as Superfect (Qiagen), liposomes, calcium phosphate transfection, polybrene, protoplast fusion, electroporation, microinjection, plasmid vectors, viral vectors, biolistic particle acceleration (the gene gun), or any of the other well known methods for introducing cloned genomic DNA, cDNA, synthetic DNA or other foreign genetic material into a host cell (see, e.g., Sambrook et al., supra).

Suitable host cells for expression of recombinant proteins include prokaryotes, yeast or higher eukaryotic cells under the control of appropriate promoters. Examples of suitable mammalian host cell lines include the COS-7 lines of monkey kidney cells, described by Gluzman (1981) Cell 23:175, and other cell lines capable of expressing an appropriate vector including, e.g., CV-1/EBNA (ATCC CRL 10478), L cells, C127, 3T3, Chinese hamster ovary (CHO), COS, NS-1, HeLa, Human embryonic Kidney Fibroblasts (HEK 293), BHK and HEK293 cell lines. Mammalian expression vectors may comprise nontranscribed elements (e.g., an origin of replication, a suitable promoter and/or an enhancer linked to the gene to be expressed, and other 5′ or 3′ flanking nontranscribed sequences) and 5′ or 3′ nontranslated sequences (e.g., necessary ribosome binding sites, a polyadenylation site, splice donor and acceptor sites, and transcriptional termination sequences). Preferred mammalian expression systems are the Chinese hamster ovary (CHO), the HEK293 and the BHK cell lines. Recombinant CHO-expressed DT fusion protein is secreted into the cell supernatant as a glycosylated protein.

Prokaryotes include gram negative or gram positive organisms, for example E. coli (e.g., BL21) or Bacilli. Higher eukaryotic cells include established cell lines of insect or mammalian origin as described below. Cell-free translation systems could also be used to produce DT fusion proteins using RNAs derived from DNA constructs. Appropriate cloning and expression vectors for use with bacterial, fungal, yeast, and mammalian cellular hosts are described, for example, by Pouwels et al., CLONING VECTORS: A LABORATORY MANUAL, Elsevier, N.Y. (1985).

Prokaryotic expression hosts may be used for expression of DT fusion proteins that do not require extensive proteolytic and disulfide processing. Prokaryotic expression vectors generally comprise one or more phenotypic selectable markers, e.g., a gene encoding a protein conferring antibiotic resistance or supplying an autotrophic requirement, and an origin of replication recognized by the host to ensure amplification within the host. Suitable prokaryotic hosts for transformation include E. coli (e.g., BL21 (DE3) CodonPlus E. coli), Bacillus subtilis, Salmonella typhimurium, and various species within the genera Pseudomonas, Streptomyces, and Staphylococcus, although other hosts may also be used.

Recombinant DT fusion proteins may also be expressed in yeast hosts such as P. pastoris. Yeast of other genera, such as Saccharomyces, Schizosaccharomyces or Kluyveromyces, may also be used. Expression in Pichia is achieved by ligation of the gene to be expressed into a bacterial shuttle vector (e.g., the pPICZ series from Invitrogen Co.), transformation of the yeast with this vector and chromosomal integration into the alcohol oxidase (AOX) locus of the yeast genome. Selection for recombinant yeast is then performed using, e.g., Zeocin (Invitrogen Co.) and protein expression is induced by the addition of methanol to the growth medium (Higgin et al., “Pichia Protocols,” METHODS IN MOLECULAR BIOLOGY, Vol. 103, Humana Press (1998)). Suitable strains of Pichia for protein expression include, e.g., the SMDI 168 Pichia strain. Expression systems based on other methodologies, such as the ESP system (Stratagene) may also be used.

Suitable yeast transformation protocols are known to one of skill in the art. An exemplary technique described by Hind et al. (1978) Proc. Natl. Acad. Sci. USA 75:1929 involves selecting for Trp+ transformants in a selective medium consisting of 0.67% yeast nitrogen base, 0.5% casamino acids, 2% glucose, 10 mg/ml adenine and 20 mg/ml uracil. Host strains transformed by vectors comprising the ADH2 promoter may be grown for expression in a rich medium consisting of 1% yeast extract, 2% peptone, and 1% glucose supplemented with 80 mg/ml adenine and 80 mg/ml uracil. Derepression of the ADH2 promoter occurs upon exhaustion of medium glucose. Crude yeast supernatants are harvested by filtration and held at 4° C. prior to further purification.

Insect (e.g., Spodoptera or Trichoplusia) cell culture systems can also be used to express recombinant polypeptides. Baculovirus systems for production of heterologous polypeptides in insect cells are reviewed, for example, by Luckow et al. (1988) BioTechnology 6:47.

D. Purification of the Fusion Proteins of the Invention

Purified DT fusion proteins may be prepared by culturing suitable host/vector systems to express the recombinant translation products of the DNAs of the present invention, which are then purified from culture media or cell extracts. For example, supernatants from systems which secrete recombinant polypeptides into culture media may be first concentrated using a commercially available protein concentration filter, such as, e.g., an Amicon or Millipore Pellicon ultrafiltration unit. Following the concentration step, the concentrate may be applied to a suitable purification matrix. For example, a suitable affinity matrix may comprise a counter structure protein (i.e., a protein to which a DT fusion protein binds in a specific interaction based on structure) or lectin or antibody molecule bound to a suitable support.

Alternatively, an anion exchange resin can be used, for example, a matrix or substrate having pendant diethylaminoethyl (DEAE) groups. The matrices can be acrylamide, agarose, dextran, cellulose, polystyrene, sepharose or other types commonly used in protein purification. Alternatively, a cation exchange step can be used. Suitable cation exchangers include various insoluble matrices comprising sulfopropyl or carboxy-methyl groups, preferably sulfopropyl groups. Gel filtration chromatography also provides a means of purifying DT fusion proteins. The fusion proteins of the invention are preferably purified by anion exchange chromatography using, e.g., monoQ columns or Q sepharose High Performance chromatography.

Affinity chromatography is another preferred method of purifying DT fusion proteins. For example, monoclonal antibodies against the DT fusion proteins may be useful in affinity chromatography purification, by using methods that are well-known in the art.

Finally, one or more reverse-phase high performance liquid chromatography (RP-HPLC) steps using hydrophobic RP-HPLC media (e.g., silica gel having pendant methyl or other aliphatic groups) may be used to further purify DT fusion protein compositions. Some or all of the foregoing purification steps, in various combinations, can also be used to provide a homogeneous recombinant protein or polypeptide.

Recombinant DT fusion proteins produced in bacterial culture may be purified by initial extraction from cell pellets, followed by one or more concentration, salting-out, aqueous ion exchange or size exclusion chromatography steps. High performance liquid chromatography (HPLC) may be used for final purification steps. Microbial cells used in expression of recombinant DT fusion proteins can be disrupted by any convenient method, including freeze-thaw cycling, sonication, mechanical disruption, or use of cell lysing agents.

Fermentation of yeast cells which express DT fusion proteins as a secreted protein greatly simplifies purification. The secreted recombinant proteins resulting from a large-scale fermentation can be purified by methods analogous to those disclosed by Urdal et al. (1984) J. Chromatog. 296:171. This reference describes two sequential, reverse-phase HPLC steps for purification of recombinant human GM-CSF on a preparative HPLC column.

Preparations of DT fusion proteins synthesized in recombinant cultures may contain non-DT cell components, including proteins, in amounts and of a character which depend upon the purification steps taken to recover the DT fusion proteins from the culture. These components are ordinarily of yeast, prokaryotic or non-human eukaryotic origin. Such preparations are typically free of other proteins which may be normally associated with the DT protein as it is found in nature in its species of origin.

In general, DT fusion proteins and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### IV. Purification of DT Fusion Proteins

Recombinant proteins of the invention can be purified from any suitable expression system, e.g., by expressing the proteins in B. anthracis and then purifying the recombinant protein via conventional purification techniques (e.g., ammonium sulfate precipitation, ion exchange chromatography, gel filtration, etc.) and/or affinity purification, e.g., by using antibodies that recognize a specific epitope on the protein or on part of the fusion protein, or by using glutathione affinity gel, which binds to GST (see, e.g., Scopes, Protein Purification: Principles and Practice (1982); U.S. Pat. No. 4,673,641; Ausubel et al., supra; and Sambrook et al., supra). In some embodiments, the recombinant protein is a fusion protein with GST or Gal4 at the N-terminus. Those of skill in the art will recognize a wide variety of peptides and proteins that can be fused to the DT fusion protein containing protein to facilitate purification (e.g., maltose binding protein, a polyhistidine peptide, etc.).

A. Purification of Proteins from Recombinant Bacteria

Recombinant and native proteins can be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of inclusion bodies. For example, purification of inclusion bodies typically involves the extraction, separation and/or purification of inclusion bodies by disruption of bacterial cells, e.g., by incubation in a buffer of 50 mM Tris/HCl pH 7.5, 50 mM NaCl, 5 mM MgCl₂, 1 mM DTT, 0.1 mM ATP, and 1 mM PMSF. The cell suspension can be lysed using 2-3 passages through a French press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. The protein of choice is separated from other bacterial proteins by standard separation techniques, e.g., ion exchange chromatography, ammonium sulfate fractionation, etc.

B. Standard Protein Separation Techniques for Purifying Proteins of the Invention

1. Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. Alternatively, the protein of interest in the supernatant can be further purified using standard protein purification techniques. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

2. Size Differential Filtration

The molecular weight of the protein, e.g., DTGM-U2, etc., can be used to isolated the protein from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The protein of choice can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

In some embodiments, the proteins are purified from culture supernatants of Bacillus or E. coli. Briefly, the proteins are purified by making a culture supernatant 5 mM in EDTA, 35% saturated in ammonium sulfate and 1% in phenyl-Sepharose Fast Flow (Pharmacia). The phenyl-Sepharose Fast Flow is then agitated and collected. The collected resin is washed with 35% saturated ammonium sulfate and the DT fusion proteins were then eluted with 10 mM HEPES-1 mM EDTA (pH 7.5). The proteins can then be further purified using a MonoQ column (Pharmacia Biotech). The proteins can be eluted using a NaCl gradient in 10 mM CHES (2-[N-cyclohexylamino]ethanesulfonic acid)-0.06% (vol/vol) ethanolamine (pH 9.1). The pooled MonoQ fractions can then be dialyzed against the buffer of choice for subsequent analysis or applications.

### V. Chemical Linkage of DT Fusion Proteins

Although certain of the methods of the invention have been described as using DT fusion proteins, it will be understood that other DT fusion protein compositions having chemically attached compounds can be used in the methods of the invention. In another embodiment, the polypeptides of the DT fusion protein, (e.g., amino acid residues 1-388 of Diphtheria toxin and GM-CSF, EGF, or IL-2) are joined via a linking group. The linking group can be a chemical crosslinking agent, including, for example, succinimidyl-(N-maleimidomethyl)-cyclohexane-1-carboxylate (SMCC). The linking group can also be an additional amino acid sequence(s), including, for example, a polyglycine linking group.

Functional groups capable of forming covalent bonds with the amino- and carboxyl-terminal amino acids or side groups of amino acids are well known to those of skill in the art. For example, functional groups capable of binding the terminal amino group include anhydrides, carbodiimides, acid chlorides, and activated esters. Similarly, functional groups capable of forming covalent linkages with the terminal carboxyl include amines and alcohols. Such functional groups can be used to bind heterologous polypeptides to DT toxin at either the amino- or carboxyl-terminus. Heterologous polypeptides can also be bound to DT through interactions of amino acid residue side groups, such as the SH group of cysteine (see, e.g., Thorpe et al., Monoclonal Antibody-Toxin Conjugates: Aiming the Magic Bullet, in Monoclonal Antibodies in Clinical Medicine, pp. 168-190 (1982); Waldmann, Science, 252: 1657 (1991); U.S. Pat. Nos. 4,545,985 and 4,894,443).

In an exemplary embodiment, the coding sequences of each polypeptide in the fusion protein are directly joined at their amino- or carboxy-terminus via a peptide bond in any order.

Alternatively, an amino acid linker sequence may be employed to separate the first and second polypeptide components by a distance sufficient to ensure that each polypeptide folds into its secondary and tertiary structures. Such an amino acid linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala, may also be used in the linker sequence. Amino acid sequences which may be usefully employed as linkers include those disclosed in Maratea et al. (1985) Gene 40:39-46; Murphy et al. (1986) Proc. Natl. Acad. Sci. USA 83:8258-8262; and in U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

Other chemical linkers include carbohydrate linkers, lipid linkers, fatty acid linkers, polyether linkers, e.g., PEG, etc. (see, e.g., Hermanson (1996) Bioconjugate Techniques).

### VI. Synthesis of DT Fusion Proteins

In one embodiment of the invention, the coding sequence of a DT fusion protein (e.g., DTGM-L1, DTGM-L2, DTGM-U2, DTGM-U3, DTEGF-L1, DTEGF-L2, DTEGF-U2, DTEGF-U3, DTIL2-L1, DTIL2-L2, DTIL2-U2, and DTIL2-U3) may be synthesized in whole or in part, using chemical methods well known in the art (see, e.g., Caruthers et al. (1980) Nuc. Acids Res. Symp. Ser. 7:215-233; Crea et al. (1980) Nuc. Acids Res. 9(10):2331; Matteucci et al. (1980) Tetrahedron Letter 21:719 (1980); and Chow et al. (1981) Nuc. Acids Res. 9(12):2807-2817).

The DT fusion polypeptide itself can be produced using chemical methods to synthesize an amino acid sequence in whole or in part. For example, peptides can be synthesized by solid phase techniques such as, e.g., the Merrifield solid phase synthesis method, in which amino acids are sequentially added to a growing chain of amino acids (see, Merrifield (1963) J. Am. Chem. Soc. 85:2149-2146). Equipment for automated synthesis of polypeptides is commercially available from suppliers such as Perkin Elmer Biosystems, Inc. (Foster City, Calif.), and may generally be operated according to the manufacturer's instructions. The synthesized peptides can then be cleaved from the resin and purified, e.g., by preparative high performance liquid chromatography (see, Creighton, PROTEINS STRUCTURES AND MOLECULAR PRINCIPLES, pp. 50-60 (1983)). The composition of the synthetic fusion polypeptides may be confirmed by amino acid analysis or sequencing (e.g., the Edman degradation procedure; see, Creighton, PROTEINS, STRUCTURES AND MOLECULAR PRINCIPLES, pp. 34-49 (1983)).

In addition, nonclassical amino acids or chemical amino acid analogs can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

### VII. Assays for Measuring Changes in Cell Growth

The administration of a functional DT fusion protein of the invention to a cell can inhibit cellular proliferation of certain cell types that overexpress MMPs and proteins of the plasminogen activation system, e.g., cancer cells, cells involved in inflammation, and the like. One of skill in the art can readily identify functional proteins and cells using methods that are well known in the art. Changes in cell growth can be assessed by using a variety of in vitro and in vivo assays, e.g., MTT assay, ability to grow on soft agar, changes in contact inhibition and density limitation of growth, changes in growth factor or serum dependence, changes in the level of tumor specific markers, changes in invasiveness into Matrigel, changes in cell cycle pattern, changes in tumor growth in vivo, such as in transgenic mice, etc.

The term “over-expressing” refers to a cell that expresses a matrix metalloproteinase, a plasminogen activator or a plasminogen activator receptor, a growth factor, or a cytokine mRNA or protein in amounts at least about twice that normally produced in a reference normal cell type, e.g., a Vero cell. Overexpression can result, e.g., from selective pressure in culture media, transformation, activation of endogenous genes, or by addition of exogenous genes. Overexpression can be analyzed using a variety of assays known to those of skill in the art to determine if the gene or protein is being overexpressed (e.g., northerns, RT-PCR, westerns, immunoassays, cytotoxicity assays, growth inhibition assays, enzyme assays, gelatin zymography, etc.). An example of a cell overexpressing a matrix metalloproteinase are the tumor cell lines, fibrosarcoma HT1080, melanoma A2058 and breast cancer MDA-MB-231. An example of a cell which does not overexpress a matrix metalloproteinase is the non-tumor cell line Vero. An example of a cells that overexpress a plasminogen activator receptor are the uPAR overexpressing cell types Hela, A2058, and Bowes. An example of a cell which does not overexpress a plasminogen activator receptor is the non-tumor cell line Vero. An example of a cells that overexpress a tissue-type plasminogen activator are cell types human melanoma Bowes and human primary vascular endothelial cells. An example of a cell which does not overexpress a plasminogen activator receptor is the non-tumor cell line Vero. An example of a cell which overexpresses GM-CSF receptors if the human leukemia cell line U397. An example of a cell which does not overexpress GM-CSF receptors is CEM-SS.

A. Assays for Changes in Cell Growth by Administration of DT Fusion Protein

One or more of the following assays can be used to identify proteins of the invention which are capable of regulating cell proliferation. The phrase “DT fusion protein constructs” refers to a DT fusion protein of the invention. Functional DT fusion protein constructs identified by the following assays can then be used to treat disease and conditions, e.g., to inhibit abnormal cellular proliferation and transformation. Thus, these assays can be sued to identify DT fusion proteins that are useful to inhibit cell growth of tumors, cancers, cancerous cells, and other pathogenic cell types.

1. Soft Agar Growth or Colony Formation in Suspension

Soft agar growth or colony formation in suspension assays can be used to identify DT fusion protein constructs which inhibit abnormal cellular proliferation and transformation. Typically, transformed host cells (e.g., cells that grow on soft agar) are used in this assay. Techniques for soft agar growth or colony formation in suspension assays are described in Freshney, Culture of Animal Cells a Manual of Basic Technique, 3rd ed., Wiley-Liss, New York (1994), herein incorporated by reference. See also, the methods section of Garkavtsev et al. (1996), supra, herein incorporated by reference.

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Administration of an active DT fusion protein to transformed cells would reduce or eliminate the host cells' ability to grow in stirred suspension culture or suspended in semi-solid media, such as semi-solid or soft. This is because the transformed cells would regenerate anchorage dependence of normal cells, and therefore require a solid substrate to grow. Therefore, this assay can be used to identify DT fusion proteins that inhibit cell growth. Once identified, such DT fusion protein constructs can be used in a number of diagnostic or therapeutic methods, e.g., in cancer therapy to inhibit abnormal cellular proliferation and transformation.

2. Contact Inhibition and Density Limitation of Growth

Contact inhibition and density limitation of growth assays can be used to identify DT fusion protein constructs which are capable of inhibiting abnormal proliferation and transformation in host cells. Typically, transformed host cells (e.g., cells that are not contact inhibited) are used in this assay. Administration of a DT fusion protein construct to these transformed host cells would result in cells which are contact inhibited and grow to a lower saturation density than the transformed cells. Therefore, this assay can be used to identify DT fusion protein constructs which are useful in compositions for inhibiting cell growth. Once identified, DT fusion protein constructs can be used in disease therapy to inhibit abnormal cellular proliferation and transformation.

Alternatively, labeling index with [³H]-thymidine at saturation density can be used to measure density limitation of growth. See Freshney (1994), supra. The transformed cells, when treated with a functional DT fusion protein, regenerate a normal phenotype and become contact inhibited and would grow to a lower density. In this assay, labeling index with [³H]-thymidine at saturation density is a preferred method of measuring density limitation of growth. Transformed host cells are treated with a DT fusion protein construct (e.g., DTGM-U2) and are grown for 24 hours at saturation density in non-limiting medium conditions. The percentage of cells labeling with [³H]-thymidine is determined autoradiographically. See, Freshney (1994), supra. The host cells treated with a functional DT fusion protein construct would give arise to a lower labeling index compared to control (e.g., transformed host cells treated with a non-functional DT fusion protein).

3. Growth Factor or Serum Dependence

Growth factor or serum dependence can be used as an assay to identify functional DT fusion protein constructs. Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. When a tumor suppressor gene is transfected and expressed in these transformed cells, the cells would reacquire serum dependence and would release growth factors at a lower level. Therefore, this assay can be used to identify DT fusion protein constructs which are able to inhibit cell growth. Growth factor or serum dependence of transformed host cells which are transfected with a DT fusion protein construct can be compared with that of control (e.g., transformed host cells which are treated with a non-functional DT fusion protein). Transformed host cells treated with a functional DT fusion protein would exhibit an increase in growth factor and serum dependence compared to control.

4. Tumor Specific Markers Levels

Tumor cells release an increased amount of certain factors (hereinafter “tumor specific markers”) than their normal counterparts. For example, tumor angiogenesis factor (TAF) is released at a higher level in tumor cells than their normal counterparts. See, e.g., Folkman, Angiogenesis and cancer, Sem Cancer Biol. (1992)).

Tumor specific markers can be assayed to identify DT fusion protein constructs, which, decrease the level of release of these markers from host cells. Typically, transformed or tumorigenic host cells are used. Administration of a DT fusion protein to these host cells would reduce or eliminate the release of tumor specific markers from these cells. Therefore, this assay can be used to identify DT fusion protein constructs are functional in suppressing tumors.

Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney Anticancer Res. 5:111-130 (1985).

5. Cytotoxicity Assay with MTT

The cytotoxicity of a particular DT fusion protein can also be assayed using the MTT cytotoxicity assay. Cells are seeded and grown to 80 to 100% confluence. The cells are then were washed twice with serum-free DMEM to remove residual FCS and contacted with a particular DT fusion protein. MTT (3-[4,5-dimethylthiazol-2-yl]-2,5-diphenyltetrazolium bromide) is then added to the cells and oxidized MTT (indicative of a live cell) is solubilized and quantified.

6. Invasiveness into Matrigel

The degree of invasiveness into Matrigel or some other extracellular matrix constituent can be used as an assay to identify DT fusion protein constructs which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used. Administration of an active DT fusion protein to these tumorigenic host cells would decrease their invasiveness. Therefore, functional DT fusion protein constructs can be identified by measuring changes in the level of invasiveness between the tumorigenic cells before and after the administration of the DT fusion protein constructs.

Techniques described in Freshney (1994), supra, can be used. Briefly, the level of invasion of tumorigenic cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

7. G₀/G₁ Cell Cycle Arrest Analysis

G₀/G₁ cell cycle arrest can be used as an assay to identify functional DT fusion protein construct. PA/LF construct administration can cause G₁ cell cycle arrest. In this assay, cell lines can be used to screen for functional DT fusion protein constructs. Cells are treated with a putative DT fusion protein construct. The cells can be transfected with a nucleic acid comprising a marker gene, such as a gene that encodes green fluorescent protein. Administration of a functional DT fusion protein would cause G₀/G₁ cell cycle arrest. Methods known in the art can be used to measure the degree of G₁ cell cycle arrest. For example, the propidium iodide signal can be used as a measure for DNA content to determine cell cycle profiles on a flow cytometer. The percent of the cells in each cell cycle can be calculated. Cells exposed to a functional DT fusion protein would exhibit a higher number of cells that are arrested in G₀/G₁ phase compared to control (e.g., treated in the absence of a DT fusion protein).

8. Tumor Growth In Vivo

Effects of DT fusion proteins on cell growth can be tested in transgenic or immune-suppressed mice. Transgenic mice can be made, in which a tumor suppressor is disrupted (knock-out mice) or a tumor promoting gene is overexpressed. Such mice can be used to study effects of DT fusion proteins as a method of inhibiting tumors in vivo.

Knock-out transgenic mice can be made by insertion of a marker gene or other heterologous gene into a tumor suppressor gene site in the mouse genome via homologous recombination. Such mice can also be made by substituting the endogenous tumor suppressor with a mutated version of the tumor suppressor gene, or by mutating the endogenous tumor suppressor, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

These knock-out mice can be used as hosts to test the effects of various DT fusion protein constructs on cell growth. These transgenic mice with a tumor suppressor gene knocked out would develop abnormal cell proliferation and tumor growth. They can be used as hosts to test the effects of various DT fusion protein constructs on cell growth. For example, introduction of DT fusion protein constructs into these knock-out mice would inhibit abnormal cellular proliferation and suppress tumor growth.

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 10⁶ cells) injected into isogenic hosts will produce invasive tumors in a high proportions of cases, while normal cells of similar origin will not. In hosts which developed invasive tumors, cells are exposed to a DT fusion protein (e.g., by subcutaneous injection). After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, functional DT fusion protein constructs which are capable of inhibiting abnormal cell proliferation can be identified. This model can also be used to identify functional versions of DT fusion proteins.

### VIII. Administration of DT Fusion Proteins

DT fusion proteins and pharmaceutical compositions comprising such proteins can be administered directly to the patient, e.g., for inhibition of cancer, tumor, or precancer cells in vivo, etc. Administration is by any of the routes normally used for introducing a compound into ultimate contact with the tissue to be treated. The compounds are administered in any suitable manner, preferably with pharmaceutically acceptable carriers. Suitable methods of administering such compounds are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)). For example, if in vivo delivery of a biologically active DT fusion protein protein is desired, the methods described in Schwarze et al. (see Science 285:1569-1572 (1999)) can be used.

The compounds, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient (“a therapeutically effective amount”), in the context of the present invention should be sufficient to effect a beneficial therapeutic response in the patient over time. The dose will be determined by the efficacy of the particular compound employed and the condition of the patient, as well as the body weight or surface area of the patient to be treated. The size of the dose also will be determined by the existence, nature, and extent of any adverse side-effects that accompany the administration of a particular compound or vector in a particular patient

In determining the effective amount of the compound(s) to be administered in the treatment or prophylaxis of cancer, the physician evaluates circulating plasma levels of the respective compound(s), progression of the disease, and the production of anti-compound antibodies. In general, the dose equivalent of a compound is from about 1 ng/kg to 10 mg/kg for a typical patient. Administration of compounds is well known to those of skill in the art (see, e.g., Bansinath et al., Neurochem Res. 18:1063-1066 (1993); Iwasaki et al., Jpn. J. Cancer Res. 88:861-866 (1997); Tabrizi-Rad et al., Br. J. Pharmacol. 111:394-396 (1994)).

For administration, compounds of the present invention can be administered at a rate determined by the LD-50 of the particular compound, and its side-effects at various concentrations, as applied to the mass and overall health of the patient. Administration can be accomplished via single or divided doses.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill in the art will readily recognize a variety of noncritical parameters that could be changed or modified to yield essentially similar results.

### Example 1

### Materials and Methods

Materials: Enzymes for DNA manipulation and modification were purchased from New England Biolabs (Beverly, Mass.). The construct pRKTGM encoding DT₃₈₈-GM and was a kind gift from Dr. Arthur E. Frankel prepared as described (Hall et al., Leukemia, 13:629-633 (1999)).

Construction of DT mutants: Mutagenic PCR was used to construct the DT mutants with the furin site replaced by MMP substrate octapeptide GPLGMLSQ (SEQ ID NO:19) in DTGM-L1 or GPLGLWAQ (SEQ ID NO:20) in DTGM-L2 and uPA substrate hexapeptide GSGRSA (SEQ ID NO:21) in DTGM-U2 and GSGKSA (SEQ ID NO:22) in DTGM-U3. The DNA fragments with desired mutations were amplified using methods known in the art and by using a universal T7 promoter primer (GTAATACGACTCACTATAGGGC) (SEQ ID NO:14) as the 5′ primer and the following mutagenic 3′ primers:

### U2

(GATTTAATGCATGACAATGAGCTACCTGCTGATCTTCCACTTCCATTTCCTGACAG GCTTG) (SEQ ID NO:15),

### U3

(GATTTATGCATGACAATGAGCTACCTGCTGATTTTCCACTTCCATTTCCTGCACAG GCTTG (SEQ ID NO:16)),

### L1

(GATTTATGCATGACAATGAGCTACCTTGACTCAACATTCCTAATGGTCCATTTCCT GCACAGGCTTG) (SEQ ID NO:17), and

### L2

(GATTTATGCATGACAATGAGCTACCTTGTGCCCATAATCCTAATGGTCCATTTCCT GCACAGGCTTG) (SEQ ID NO:18). For each of the 3′ mutagenic primers, antisense sequences encoding the mutated protease sites are underlined and the native Nsi I restriction site is in italics.
The amplified fragments were digested by NsiI and XbaI, the resulting fragments were used to replace the XbaI-NsiI fragment in pRKDTGM, resulting constructs pRKDTGM-U2, pRKDTGM-U3, and pRKDTGM-L1, encoding the desired fusion proteins, respectively.

Expression and Purification of DTGM, DTGM-U2, DTGM-U3, DTGM-L1, and DTGM-Fu: To express DTGM, DTGM-U2, DTGM-U3, DTGM-L1, and DTGM-Fu, expression plasmids containing the appropriate constructs were transformed into BL21, and grown in Superbroth and induced with 1 mM isopropyl-D-thiogalactopyranoside (IPTG) for 3 h at 37° C. The DT fusion proteins were purified by ammonium sulfate precipitation followed by monoQ column (Pharmacia Biotech) chromatography, as described previously (Varughese, M., et al., Infect Immun, 67:1860-1865 (1999)).

Cytotoxicity assay with MTT: Cytotoxicity of DTGM, DTGM-U2, DTGM-U3, and DTGM-Fu the test cells (U397 and CEM-SS) were performed in 96-well plates. Cells were properly seeded into 96-well plates so that they reached 80 to 100% of confluence the next day. The cells were washed twice with serum-free DMEM to remove residual FCS. In some cases, the cells were incubated in the presence of pro-uPA for one hour before the addition of the DT fusion proteins. Then serially diluted DTGM, DTGM-U2, DTGM-U3, and DTGM-Fu (from 0 to 1000 ng/ml) in serum-free DMEM were added to the cells to a total volume of 200 μl/well and the cells were incubated for 48 hours. Cell viability was assayed by adding 50 μl of 2.5 mg/ml MTT (3-[4,5-dimethylthiazol-2-yl]-2,5-diphenyltetrazolium bromide). The cells were incubated with MTT for 45 min at 37° C., live cells oxidized MTT to blue dye precipitated in cytosol while dead cells remained colorless. Then removed media and solubilized the blue precipitate with 100 μl/well of 0.5% (w/v) SDS, 25 mM HCl, in 90% (v/v) isopropanol. The plates were vortexed and the intensity of the oxidized MTT read at 570 nm using the microplate reader.

Cytotoxicity assay with MTS/PMS: Cytotoxicity of DTGM, DTGM-U2, DTGM-U3, and DTGM-Fu the test cells (U397 and CEM-SS) were performed in 96-well plates. Cells were properly seeded into 96-well plates so that they reached 80 to 100% of confluence the next day. The cells were washed twice with serum-free DMEM to remove residual FCS. In some cases, the cells were incubated in the presence of pro-uPA for one hour before the addition of the DT fusion proteins. Then serially diluted DTGM, DTGM-U2, DTGM-U3, and DTGM-Fu (from 0 to 1000 ng/ml) in serum-free DMEM were added to the cells to a total volume of 200 μl/well and the cells were incubated for 48 hours. Cell viability was assayed by adding MTS/PMS (3-(4,5-dimethylthiazol-2-yl)-5-(3-carboxymethoxyphenyl)-2-(4-sulfophenyl)-2H-tetrazolium, inner salt/phenazine methosulfate) as described in Buttke et al., J Immunol Methods 57(1-2):233-40 (1993).

### Example 2

### Construction of Mutant PA with Matrix Metalloproteinase Cleavage Sites

Human GM-CSF was recombinantly fused to the C-terminus of modified DT388. The table represents the sequence modified in the furin sensitive surface loop of DTGM that generate cleavage sites recognized by furin, uPA, or MMP as indicated. To generate DTGM-U2, the native furin cleavage site was replaced by GSGRSA, a urokinase plasminogen cleavage site. To generate DTGM-U3, the native furin cleavage site was replaced by GSGKSA, a urokinase plasminogen cleavage site. To generate DTGM-L1, the native furin cleavage site was replaced by GPLGMLSQ, a matrix metalloproteinase cleavage site.

### Example 3

### Production of DT Fusion Proteins

pRKDTGM encoding the modified diphtheria toxin GM-CSF fusion protein was transformed into E. coli (BL2) cells which were then incubated at 37° C. in Superbroth, and induced with 1 mM IPTG for 3 hours. Cells were lysed, inclusion bodies were isolated, washed with TES buffer with Triton X-100, and denatured in guanidine-HCl with DTT. Soluble proteins were refolded for 48 hours in buffer containing L-arginine and glutathione. The isolated protein was then dialyzed, filter sterilize, and purified over columns.

### Example 4

### Cytotoxicity of DT Fusion Proteins

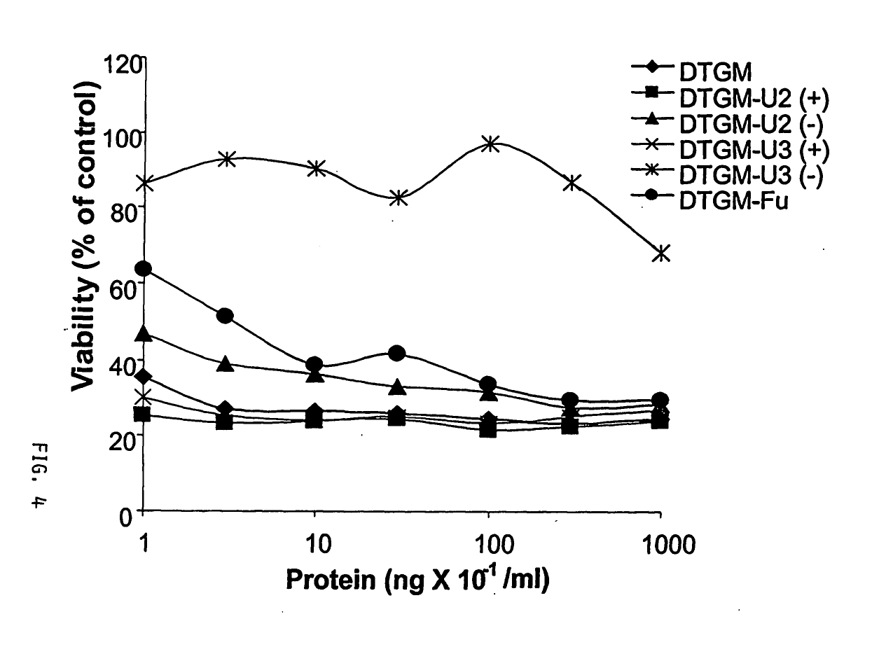

FIG. 4 illustrates data showing cytotoxicity of mutant DTGM fusion proteins to U397 human leukemia cells, a GM-CSF receptor positive cell line, were plated in a 96 well plate at 4×10⁴ cells per well per day prior to treatment with mutant DTGM fusion proteins. Cells were incubated in the presence or absence of pro-uPA for one hour before the addition of mutant DTGM fusion proteins. All cells were incubated with serial dilutions of DTGM, DTGM-U2, DTGM-U3 or DTGM-Fu fusion proteins for 48 hours at 37° C. MTS/PMS was added to determined cell viability at 48 hours. The results are shown in FIG. 4.

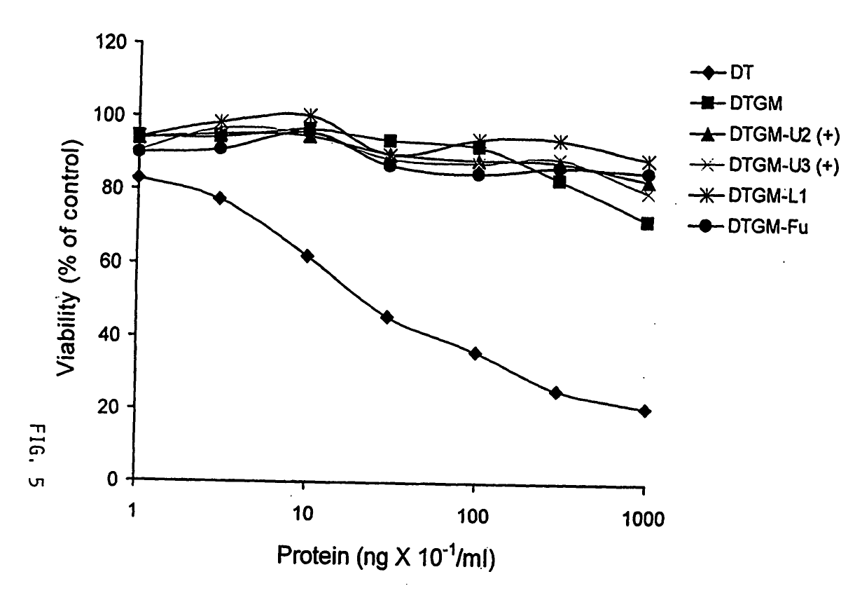

CEM-SS cells, a GM-CSF receptor negative cell line were incubated with serial dilutions of DT, DTGM, DTGM-U2, DTGM-U3, DTGM-L1, and DTGM-Fu fusion proteins for 48 hours at 37° C. Some cells were preincubated with pro-uPA (100 ng/ml) for one hour before addition of DTGM proteins (DTGM-U2 and DTGM-U3). All cells were incubated with serial dilutions of DT, DTGM, DTGM-U2, DTGM-U3, DTGM-L1, and DTGM-Fu fusion proteins for 48 hours at 37° C. MTS/PMS was added to determined cell viability at 48 hours. The results are shown in FIG. 5.

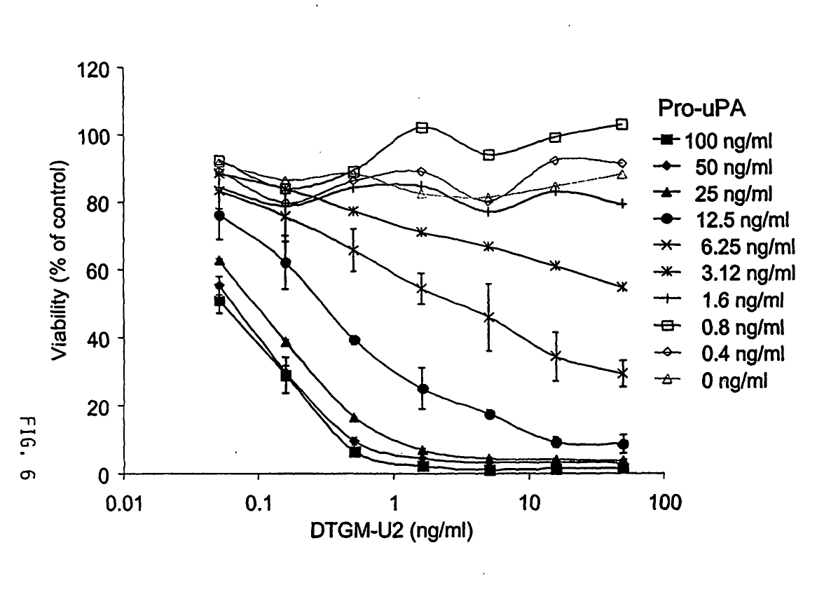

Human leukemia U937 cells were treated with multiple concentrations of DTGM-Us and pro-uPA for 48 hours. Then MTT was added to determine cell viability. Live cells converted MTT to blue dye, which precipitated in cytosol, while dead cells remained colorless. The cytotoxicity of DTGM-U2 to U937 cells was dependent on the presence of pro-uPA. The results are shown in FIG. 6.

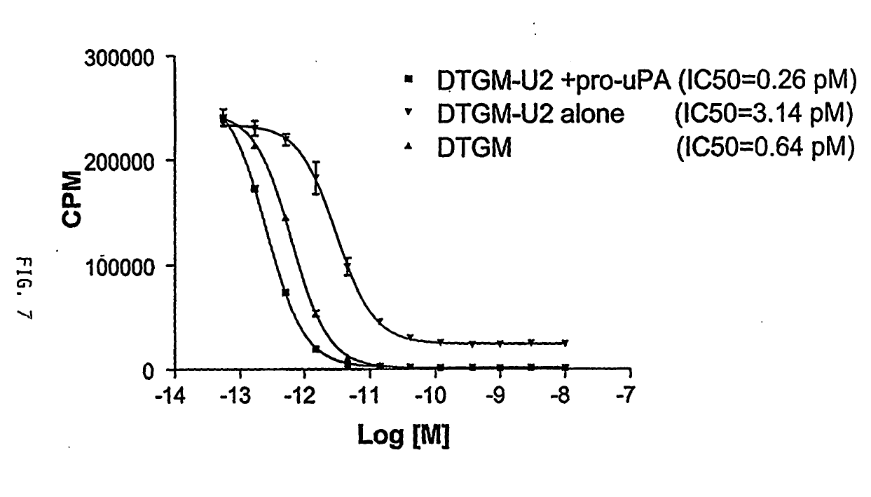

Human leukemia TF1-vRAF cells were treated with various concentrations of DTGM alone or DTGM-U2 with and without exogenous human pro-uPA (50 ng/ml) for 48 hours. Thymidine incorporation assays were then performed to determine the cytotoxicity of the mutant DT fusion proteins to the cells. Killing was observed even without exogenous pro-uPA. In the presence of pro-uPA, DTGM-U2 was even more cytotoxic to the leukemia cells. The results are shown in FIG. 7.

### Example 5

### DTGM-U2 is Selectively Cytotoxic to Human Acute Myeloid Leukemia Cells

The cytotoxicity of DTGM-U2 was tested against several AML cells lines, including TF1-vRaf AML cell line (proliferation inhibition assay; IC50=3.14 pM), and this toxicity was greatly inhibited following pretreatment with anti-uPA and anti-GMCSF antibodies. The activity of this toxin was then tested on a larger group of 13 human AML cell lines and 5 of the 13 cell lines were sensitive to DTGM-U2. An additional 5 of the 13 cell lines became sensitive when exogenous pro-uPA was added. DTGM-U2 was less toxic to normal cells expressing uPAR or GMCSFR alone, i.e. HUV-EC and peripheral macrophages, respectively. These results indicate that DTGM-U2 may be a selective and potent agent for the treatment of patients with AML.

DTGM-U2: The expression plasmid coding for uPA-activated DTGM, i.e., DTGM-U2 or DTU2GMCSF, was constructed and expressed as described in Example 1 above.

Other toxins used. DT₃₈₈GMCSF (i.e., DTGM) was produced as described in Frankel et al., Protein Expr. Purif. 16(1):190-201 (1999). DTAT consists of the translocation and catalytic domains of DT fused to the N-terminal domain of uPA which targets DT to uPAR expressing cells (see, e.g., Andreasen et al., Cell. Mol. Life. Sci. 57(1): 25-40 (2000). DTAT was a generous gift of Daniel Vallera at the University of Minnesota.

Cells and cell lines. Human AML cell lines HL60, U937, ML1, ML2, ML17, Monomac 1, Monomac 6, CTV-1, KG-1, Sig M5 and TF1v-Src were grown as described in e.g., Dano et al., APMIS 107(1): 120-7 (1999). The two other human AML cell lines used in this study, TF1-vRaf and TF1-vSrc were cultured as described in, e.g., Kiser et al., Leukemia 15(5):814-8 (2001). Human umbilical vein endothelial cells (HUV-EC) were purchased from ATCC and cultured according to the directions provided. Peripheral monocytes were obtained and isolated from healthy adult individuals.

Cell line sensitivity to DTGM-U2. Aliquots of 1 cells were incubated in 100 μl medium (same as that used to grow the cells) in Costar 96-well flat-bottomed plates in duplicate. Exogenous human pro-uPA (American Diagnostica, Stamford Conn.) was added to 36 wells of each duplicate plate (100 ng/ml in each well). Fifty microliters of DTGM-U2 in medium was added to each column to yield concentrations ranging from 0.1 to 10,000 pM, and the cells were incubated at 37° C./5% CO₂ for 50 h. 1 μCi ³H-thymidine (NEN DuPont, Boston, Mass.) in 50 μl medium was added to each well, and incubation continued for an additional 18 h at 37° C./5% CO₂. Cells were then harvested using a Skatron Cell Harvester (Skatron Instruments, Lier, Norway) on to glass fiber mats and counts per minute (CPM) of incorporated radiolabel were counted using an LKB liquid scintillation counter gated for ³H. The IC₅₀ was defined as the concentration of toxin which inhibited thymidine incorporation by 50% compared to control wells. The percent maximal ³H-thymidine incorporation was plotted versus the log of the toxin concentration, and non-linear regression with a variable slope sigmoidal dose response curve was generated along with IC₅₀ using GraphPad Prism software (GraphPad Software, San Diego, Calif.). All assays were performed at least twice with inter-assay range of ≦30% for IC₅₀.

Monocytes and HUV-EC sensitivity to DTGMU2. Normal cell sensitivity to DTGM-U2 was determined using the same cytotoxicity assay described in the previous section (cell line sensitivity to DTGM-U2) with the following differences: Both normal monocytes and HUV-EC were plated 24 hours prior to incubation with DTGM, DTGM-U2 and DTAT (only for HUV-EC). Exogenous pro-uPA was not added in these assays. Cells were incubated with the toxins for 5 hours in the case of peripheral monocytes and 48 hours for HUV-EC. The efficacy of DTGM-U2, DTGM and DTAT on HUV-EC was determined by ³H-thymidine incorporation inhibition assay; 50 μl of a 10 μCi/ml ³H-thymidine solution was added to each well, and the cells were harvested 24 hours after the addition of ³H-thymidine. The efficacy of DTGM-U2 and DTGM was determined by ³H-leucine incorporation inhibition for normal monocytes; 100 μl of a 20 μCi/ml ³H-leucine solution (NEN DuPont, Boston, Mass.) was added to each well, and the cells were harvested 6 hours following the addition of ³H-leucine.

GMCSFR expression GMCSFR expression levels were determined in all cell lines as described previously.¹⁵ In short, aliquots of 3 to 5×10⁶ cells were mixed with varying amounts of ¹²⁵I Bolton-Hunter labeled human GM-CSF (80 to 120 μCi/μg, NEX249; DuPont, Boston, Mass.) with or without excess (1,500 ng) cold GM-CSF (Immunex, Seattle, Wash.). Cells were incubated at 37° C. for 1 hour and then layered over a 200 μL oil phthalate mixture. After centrifugation at 12,000 rpm for 1 minute in a microfuge at room temperature, both pellets and supernatants were saved and counted in an LKB-Wallac 1260 Multi-gamma counter (Turku, Finland) gated for ¹²⁵I with 50% counting efficiency. Background CPM was calculated by linear extrapolation from incubations with excess cold GM-CSF. Experiments were performed in duplicate. Receptor number/cell (Bₘₐₓ) as well as dissociation constant (Kd) were calculated using the Graph Pad Prism software.

uPAR expression uPAR expression levels were determined following the same protocol used for the determination of GMCSFR levels (see, e.g., Boyum, Nature 204:793-4 (1964)). The amino-terminal fragment (ATF) of uPA (amino acids 1-133) was a generous gift from Dr. Michael Ploug. ¹²⁵I-labeling of the ATF was done in Iodogen coated tubes; 8 μg of ATF were incubated with 0.5 mCi of ¹²⁵I and 10 μl binding buffer for 15 minutes. The reaction was then stopped with the addition of excess amounts of binding buffer and 10 μl of the reaction mixture were added to 900 μl RIA buffer and 100 μl TCA. The mixture was then centrifuged at 2000 rpm for 10 minutes, and the percentage of labeled ATF as well as the specific activity of labeled ATF was calculated by determining the amount of radioactivity in the pellet versus the amount of radioactivity in the supernatant. ¹²⁵I-labeled ATF was separated from free 125, on a desalting column. Calculations and analysis were done using the Graph Pad Prism software.

uPA and PAI-1 levels uPA and PAI-1 levels were determined using enzyme-linked immunosorbent assay (ELISA) kits (American Diagnostica), and the assays were done according to the description provided in the each assay kit. For each cell line 100 μl of cell culture supernatant (cell density>10⁶ cells/ml) were used, in duplicates, in each ELISA assay. Both these kits detect total levels of uPA and PAI-1.

Blocking assays Blocking assays were performed to test the ability of specific anti-GMCSF and anti-uPA antibodies to block the killing of the sensitive cell lines by DTGM-U2. The same thymidine incorporation inhibition assays were performed as described above but without the addition of pro-uPA. In the blocking assays, 10 μg/ml of monoclonal anti-uPA antibody (American Diagnostica) or 2 μg/ml of monoclonal anti-GMCSF antibody (Oncogene Research, San Diego Calif.) were added to the cells 2 hours before the addition of DTGM-U2. The rest of the assay proceeded as described earlier.

DTU2GMCSF expression and purification. Recombinant protein was expressed in E. coli, purified from inclusion bodies with a yield of 16 mg/L of bacterial culture and a purity of >95% by Coomassie stained SDS-PAGE. DTGM-U2 is refolded as described previously for the refolding of DTGM (see, e.g., Frankel et al., Protein Expr. Purif. 16(1):190-201 (1999)). The percent of properly folded protein recovered from the inclusion bodies is about 25%.

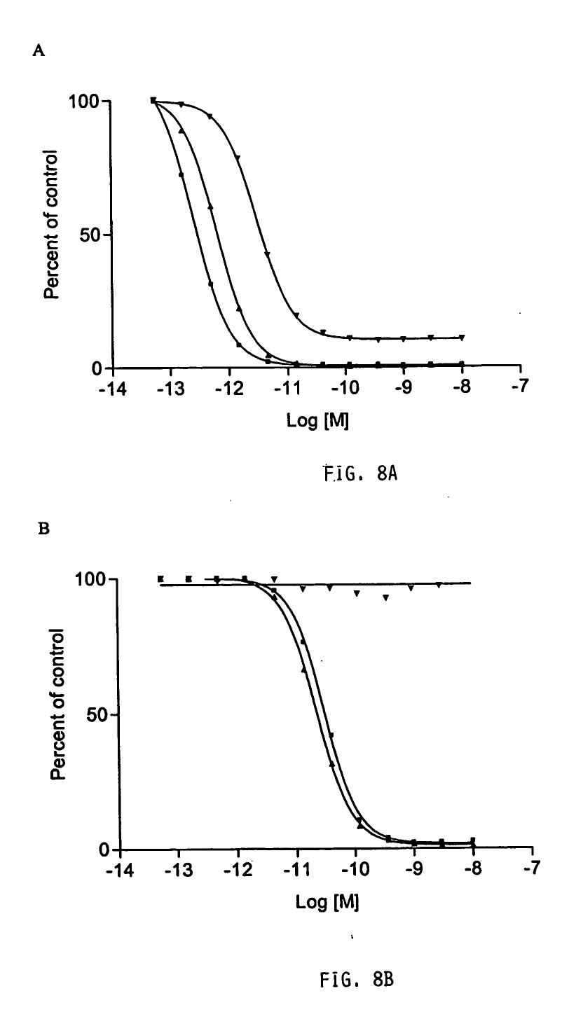

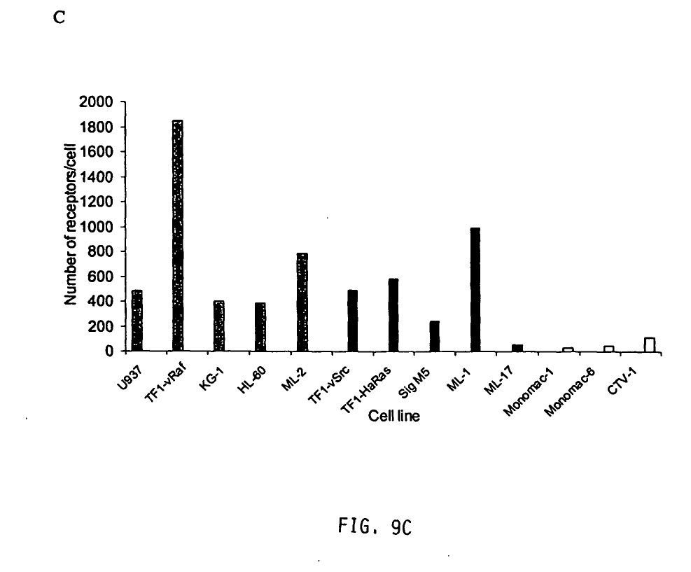

DTGM-U2 cytotoxicity. To determine the biological activity of DTGM-U2 we tested the cytotoxicity of this fusion protein on a panel of 13 AML cell lines (FIG. 11). Tritiated thymidine incorporation inhibition assays were done using DTGM-U2 with or without the addition of exogenous pro-uPA as well as using DTGM (i.e., All the cell lines tested had a high sensitivity to DTGM except CTV-1 which had an IC₅₀ for DTGM >4000 pM. This cell line was also not sensitive to DTGM-U2 with or without the addition of exogenous pro-uPA. Five out of thirteen cell lines, namely U937, TF1-vRaf, KG-1, HL60 and ML-2, were highly sensitive to DTGM-U2 without the addition of exogenous pro-uPA (IC₅₀=3.14-34.7 μM) (FIG. 11, FIG. 8A). When these cells where coincubated with DTGM-U2 and exogenous pro-uPA, the cytotoxicity of DTGM-U2 significantly increased and became similar to that of DTGM (IC₅₀=2.6-3.5 μM). Another five out of thirteen cell lines, namely TF1-vSrc, TF1-HaRas, Sig M5, ML-1 and ML17 were not sensitive to DTGM-U2. These cell lines, however, when coincubated with both DTGM-U2 and exogenous pro-uPA had significantly increased sensitivity to DTGM-U2 (IC₅₀=0.55-42 pM) becoming similar to the sensitivity of these cells to DTGM (FIG. 11, FIG. 8B). The remaining three cell lines—Monomac 1, Monomac 6 and CTV-1 were not sensitive to DTGM-U2 even when exogenous pro-uPA was added. Monomac 1 and Monomac 6 were sensitive to DTGM (IC₅₀=3.2 and 68.3 pM respectively), while CTV-1 was not sensitive even to DTGM (FIG. 11, FIG. 8C).

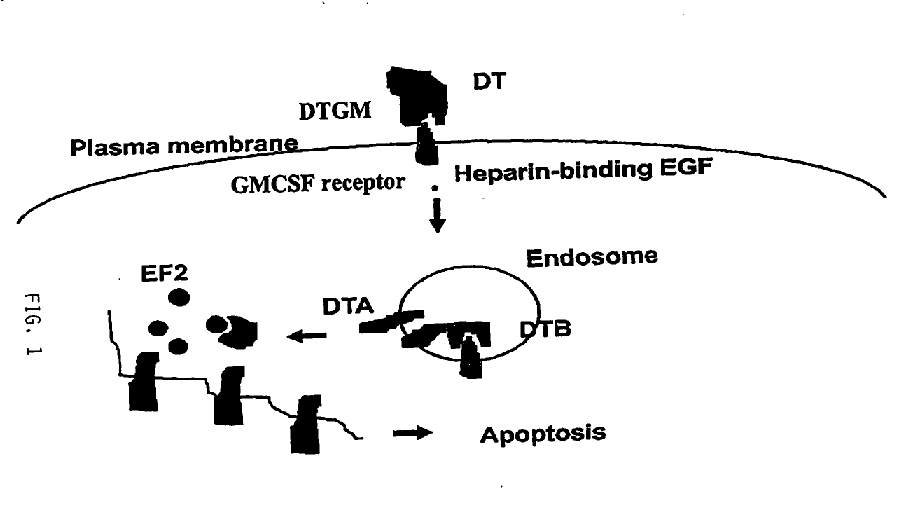

Inhibition assays. To show the requirement for the presence of GMCSFR and an active uPA/uPAR protease system for DTGM-U2 toxicity, we inhibited each of these components separately and looked at the effects of these inhibitions on the efficiency of DTGM-U2. We inhibited the uPA/uPAR system by pre-incubating TF1-vRaf cells with a specific monoclonal antibody directed against an epitope close to the active site of double-chain uPA. This antibody binds and inactivates all forms of uPA (single chain pro-uPA and double chain uPA), and it inhibits the proteolytic activity of active double chain uPA even in its uPAR bound form. Incubation of the cells with 10 μg/ml anti-uPA antibody increased the IC₅₀ of DTGM-U2 by 106 fold (IC₅₀=0.62 μM) as compared to DTGM-U2 alone (IC₅₀=16.6 pM). (FIG. 1D). We separately inhibited the binding of DTGM-U2 to its GMCSFR, by co-treating cells with an anti-GMCSF monoclonal antibody that targets an epitope near the GMCSFR binding site on GMCSF and subsequently blocks binding of GMCSF to its receptor. Incubation of the cells with 2 μg/ml anti-GMCSF increased the IC₅₀ of DTGM-U2 by 200 fold (IC₅₀=400 pM) as compared to the IC₅₀ of DTGM-U2 alone (2.3 pM) (FIG. 8D).

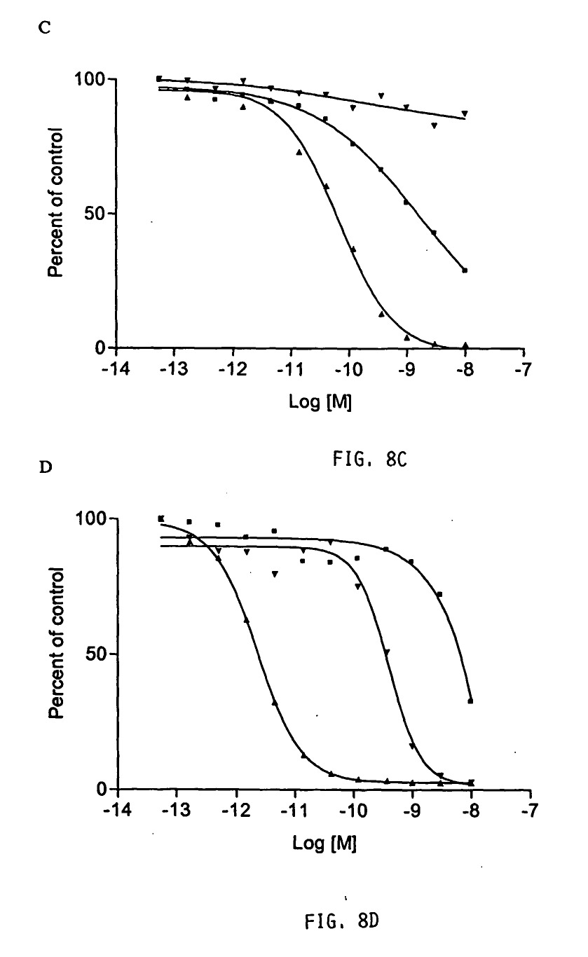

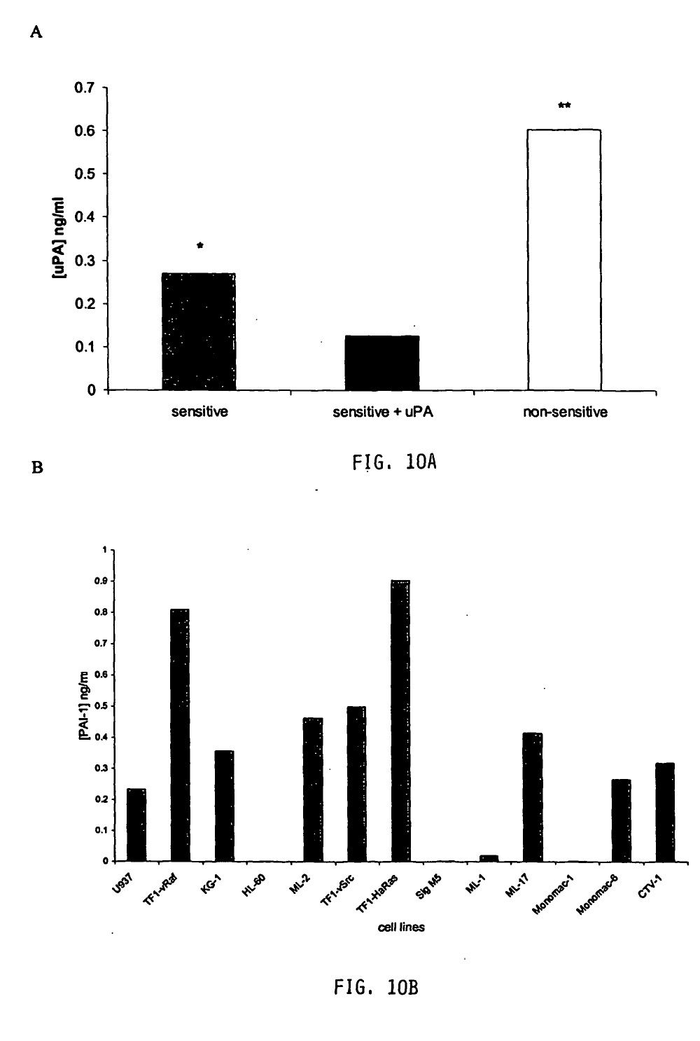

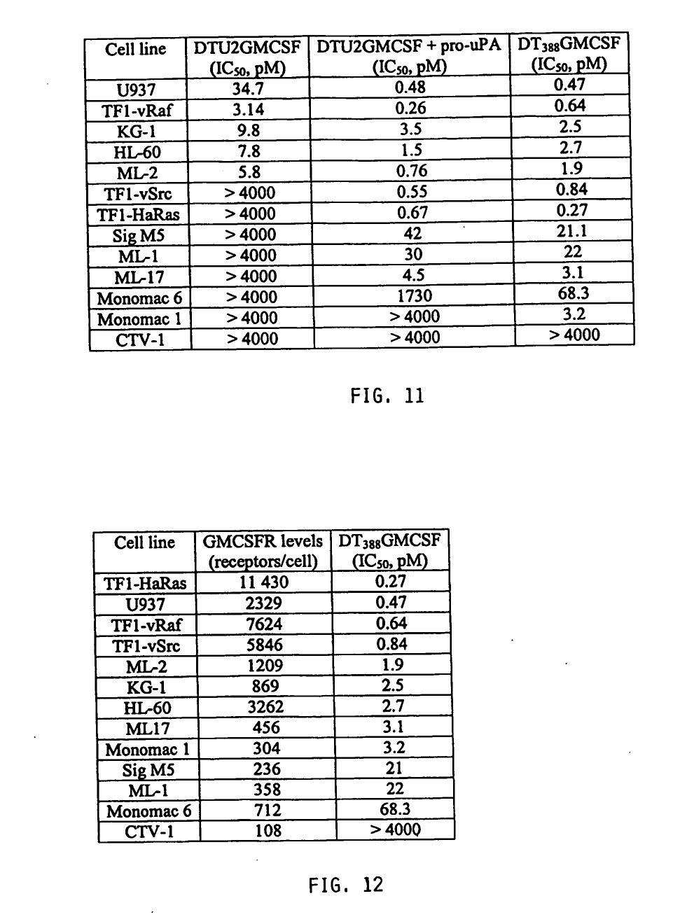

Receptor levels. To determine the minimal expression levels of GMCSFR and uPAR needed for DTGM-U2 to be active, we determined the expression levels of these two receptors in the 13 AML cell lines tested for cytotoxicity, and correlated these expression levels to the sensitivity of each cell line for DTGM-U2. GMCSFR levels varied between 108 receptors/cell for CTV1 and 11430 receptors/cell for TF1-HaRas (FIG. 9A). The low GMCSFR expressing CTV1 cells were resistant to both DTGM and DTGM-U2. GMCSFR density correlated with the sensitivity of each cell line to DTGM (FIG. 12). Among the 12 cell lines that were sensitive to DTGM, Sig M5 had the lowest number of GMCSFR (236 receptors/cell). Therefore the minimum number of GMCSFR required for a cell to be sensitive to DTGM in our survey was 236 receptors/cell. The two cell lines that were sensitive to DTGM but not sensitive to DTGM-U2 had very low levels of uPAR expression. uPAR levels in these cell lines were 33 receptors/cell for Monomac 1 and 45 receptors/cell for Monomac 6. On the other hand, the 10 cell lines that were sensitive to DTGM-U2 with or without the addition of pro-uPA had high levels of uPAR expression. uPAR levels varied between 387 and 1848 receptors/cell for the five cell lines that were sensitive to DTGM-U2 alone (FIG. 13). Receptor levels varied between 54 and 991 receptors/cell for the five cell lines that were sensitive to DTGM-U2 only when pro-uPA was added (FIGS. 9B and 9C, FIG. 13). The sensitive cell line with the lowest uPAR expression levels (ML-17) had 54 receptors/cell and was sensitive to DTGM-U2 when excess uPA was added. Therefore the minimum expression levels of uPAR required for the cells to be sensitive to DTGM-U2 in our survey was 54 receptors/cell. uPAR expression levels in the DTGM-U2-resistant cell lines were significantly lower than expression levels in all other cells lines. On the other hand, uPAR expression levels in cell lines sensitive to DTGM-U2 did not distinguish cell lines requiring the addition of exogenous pro-uPA from those not requiring exogenous pro-uPA

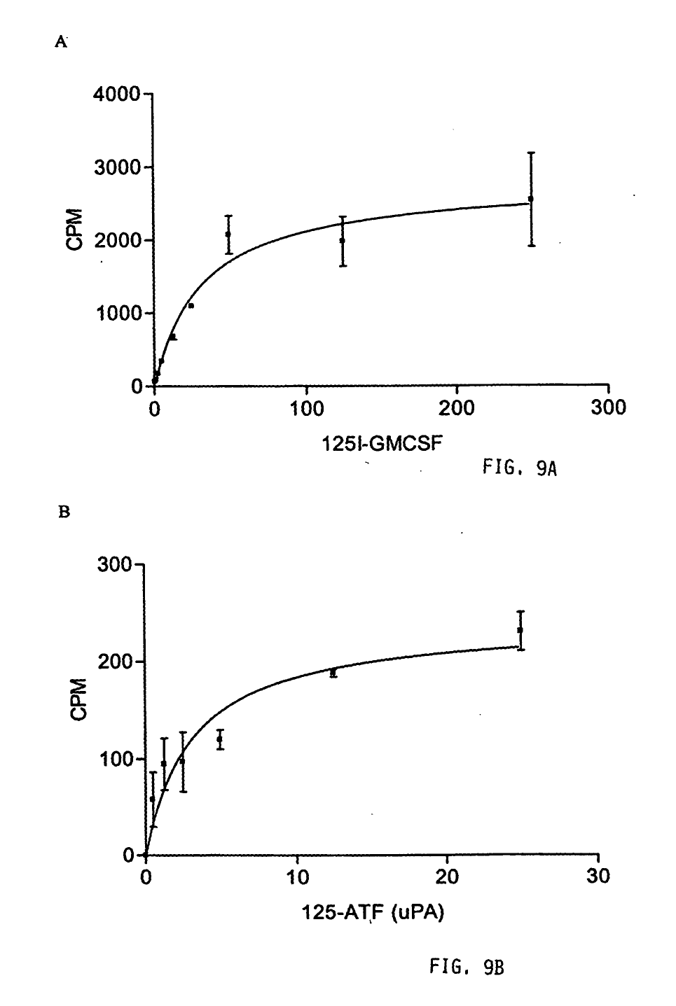

uPA and PAI-1 levels. We determined total uPA levels in cultures with densities comprised between 1.5 and 2 million cells/ml, using an ELISA kit that detects all forms of uPA, i.e. inactive single chain pro-uPA as well as double chain active uPA. Total uPA levels in the 13 cell lines tested varied between 0.1 and 0.93 ng/ml (FIG. 13). The highest levels of total uPA were observed in the cell lines that are resistant to DTGM-U2, these levels were: 0.93 ng/ml for Monomac 1, 0.55 ng/ml for Monomac 6 and 0.32 ng/ml for CTV1. This result indicates that in the absence of adequate uPAR expression cells were resistant to DTGM-U2 even in the presence of relatively high levels of total uPA. Further, cells that were sensitive to DTGM-U2 without the addition of pro-uPA had significantly higher total uPA levels than cell lines that needed the addition of exogenous pro-uPA to be sensitive (FIG. 10A). PAI-1 levels varied between 0.018 and 0.903 ng/ml and did not correlate with the sensitivity of each cell line to DTGM-U2. Three cell lines, namely HL60, Sig M5 and Monomac 1 did not have any detectable levels of PAI-1 (FIG. 10B). However, this finding does not rule out the possibility of PAI-1 activity being a major regulator of DTGM-U2 efficacy in vivo.

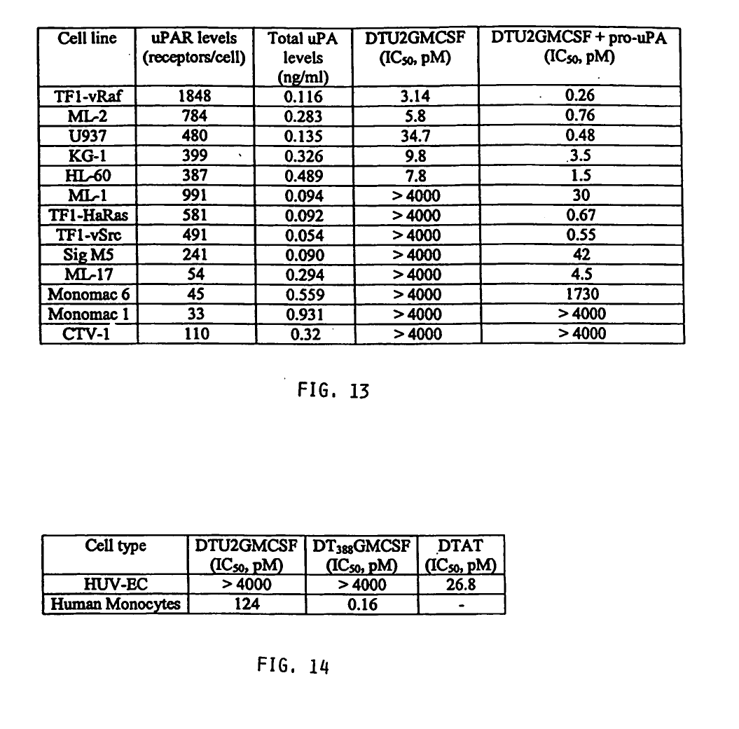

DTGM-U2 toxicity to normal cells. In order to determine the toxicity of DTGM-U2 to normal cells we tested the sensitivity of both peripheral monocytes and human umbilical vein endothelial cells (HUV-EC) to DTGM-U2 and DTGM. Normal peripheral monocytes, with GMCSFR but low uPA/uPAR protease activity, were 10³-fold less sensitive to DTGM-U2 than to DTGM. HUV-EC, cells that express uPAR but low or absent GMCSFR, were resistant to both DTGM and DTGM-U2 but sensitive to DTAT (IC₅₀=26.8 pM). Thus, DTGM-U2 is a dual specificity cytotoxin requiring the presence of GMCSFR and an active uPA/uPAR protease system (FIG. 14).

The first generation of DT fusion toxins (*i.e., DT₃₈₈GMCSF, DT₃₈₈IL3 and DTAT) for AML have only a single targeting moiety—the ligand and were potentially toxic to normal tissues bearing their respective receptors—GMCSFR, IL3R or uPAR (see, e.g., Frankel et al., Protein Expr. Purif. 16(1):190-201 (1999); Ramage et al., Leukemia Research 27(1): 79-84 (2003); and Urieto et al., Protein Expr Purif. 33(1): 123-33 (2004)). Clinical testing of the AML fusion toxin DTGM confirmed clinical efficacy but with associated damage to GMCSFR containing normal cells. This led to significant liver injury. We replaced DT ₁₆₃RVRRSV₁₇₀ with the uPA cleavage sequence ₁₆₃GSGRSA₁₇₀ in DTGM, to generate a new AML fusion toxin DTGM-U2 that would retain potency but have enhanced AML specificity. The new dual specificity fusion toxin DTGM-U2 was produced in the same yields and purity as DTGM. The protein remained stable and biologically active after storage at −80° C. for over one month. Further, DTGM-U2 remained fairly stable after incubation with serum at 37° C. for 48 hours (IC₅₀=12.8 pM as compared to an IC₅₀=6.3 pM for freshly thawed DTGM-U2 in a tritiated thymidine incorporation inhibition assay on HL60 cells).

Remarkably, DTGM-U2 showed potency similar to DTGM when exogenous single chain pro-uPA was added. This suggests that uPAR is expressed in adequate levels on most AML cell lines. The deficiency of uPA, in some cases, may be an artifact of in vitro culture or may occur during cell line generation. In vivo, there may be higher concentrations of uPA in the tumor environment (see, e.g., Tapiovaara et al., Blood 82: 914-919 (1993). Even without exogenous pro-uPA, 5/13 cell lines (39%) were killed by DTGM-U2. Thus, a significant fraction of patients may have disease sensitive to this fusion toxin.

DTGM-U2 is a dual specificity fusion toxin. Blocking assays, normal tissue toxicities and cell line receptor studies show that both uPA/uPAR and GMCSFR are required for DTGM-U2 toxicity. Several groups have made toxins targeting more than one tumor cell receptor (see, e.g., Todhunter et al., Protein Eng Des Sel. (2):157-64 (2004) and Schmidt et al., Int J Cancer. 65(4): 538-46 (1996)). In most cases, the toxins had two ligands and thus were able to intoxicate cells with either ligand, e.g. IL13 receptor and uPAR (see, e.g., Todhunter et al., Protein Eng Des Sel. (2):157-64 (2004)) and EGF receptor and erbB-2 (see, e.g., Schmidt et al., Int J Cancer. 65(4): 538-46 (1996)). Hence, while the new molecules were able to bind a larger range of tumor cells, they had less rather than more specificity.

We provide a proof of principle for the efficacy and increased specificity of dual specificity fusion toxins. Many of the receptor targets for fusion toxins have limited specificity, hence addition of a requisite toxin processing step with tumor selective proteases may yield safer active fusion toxins for cancer treatment.

All publications, patent applications, and accession numbers cited in this specification are herein incorporated by reference as if each individual publication, patent application, or accession number were specifically and individually indicated to be incorporated by reference.

1. A nucleic acid encoding a Diphtheria toxin fusion protein comprising
(1) residues 1-388 of Diphtheria toxin, wherein the native furin cleavage site has been substituted for a cleavage site for a matrix metalloproteinase or a plasminogen activator; and
(2) a heterologous polypeptide, wherein the heterologous polypeptide specifically binds to a protein overexpressed on the surface of a cell.

2. The nucleic acid of claim 1, wherein the matrix metalloproteinase is selected from the group consisting of MMP-2 (gelatinase A), MMP-9 (gelatinase B) and membrane-typel MMP (MT1-MMP).

3. The nucleic acid of claim 1, wherein the plasminogen activator is selected from the group consisting of tissue plasminogen activator (t-PA) and urokinase plasminogen activator (u-PA).

4. The nucleic acid of claim 1, wherein the matrix metalloproteinase cleavage sites are GPLGMLSQ (SEQ ID NO: 19) and GPLGLWAQ (SEQ ID NO: 20).

5. The nucleic acid of claim 1, wherein the plasminogen activator cleavage site is selected from the group consisting of QRGRSA (SEQ ID NO: 23), GSGRSA (SEQ ID NO: 21) and GSGKSA (SEQ ID NO: 22).

6. The nucleic acid of claim 1, wherein the protein overexpressed on the surface of a cell is a receptor.

7. The nucleic acid of claim 1, wherein the heterologous polypeptide comprises a cytokine.

8. The nucleic acid of claim 1, wherein the heterologous polypeptide comprises a growth factor.

9. The nucleic acid of claim 1, wherein the heterologous polypeptide is a member selected from the group consisting of: Il-2, GM-CSF, and EGF.

10. The nucleic acid of claim 1, comprising the nucleotide sequence set forth in SEQ ID NO: 2, 3, 4, 5, 6, 7, 8, 9, 10, 11, 12, or 13.

11. A vector comprising the nucleic acid of claim 1.

12. The nucleic acid of claim 6, wherein the cell is a cancer cell.

13. The nucleic acid of claim 7, wherein the heterologous polypeptide comprises GM-CSF.

14. The nucleic acid of claim 7, wherein the heterologous polypeptide comprises IL-2.

15. The nucleic acid of claim 8, wherein the heterologous polypeptide comprises EGF.

16. A nucleic acid encoding a Diphtheria toxin fusion protein comprising
(1) residues 1-388 of Diphtheria toxin, wherein the native furin cleavage site has been substituted for a cleavage site for a urokinase a plasminogen activator; and
(2) GM-CSF.

17. A polypeptide encoded by the nucleic acid of claim 1.

18. A polypeptide encoded by the nucleic acid of claim 10.

19. A polypeptide encoded by the nucleic acid of claim 16.

20. A host cell comprising the vector of claim 11.

21. The nucleic acid of claim 12, wherein the cancer is leukemia.

22. The nucleic acid of claim 12, wherein the cancer is acute myelogenous leukemia.

23. A pharmaceutical composition comprising the protein of claim 18 and a pharmaceutically acceptable carrier.

24. A method of treating cancer, the method comprising administering to a subject a Diphtheria toxin fusion protein comprising
(1) residues 1-388 of Diphtheria toxin, wherein the native furin cleavage site has been substituted for a cleavage site for a matrix metalloproteinase or a plasminogen activator; and
(2) a heterologous polypeptide, wherein the heterologous polypeptide specifically binds to a protein overexpressed on the surface of a cell.

25. The method of claim 24, wherein the matrix metalloproteinase is selected from the group consisting of MMP-2 (gelatinase A), MMP-9 (gelatinase B) and membrane-typel MMP (MT1-MMP).

26. The method of claim 24, wherein the plasminogen activator is selected from the group consisting of t-PA and u-PA.

27. The method of claim 24, wherein the matrix metalloproteinase cleavage sites are GPLGMLSQ (SEQ ID NO: 19) and GPLGLWAQ (SEQ ID NO: 20).

28. The method of claim 24, wherein the plasminogen activator cleavage site is selected from the group consisting of QRGRSA (SEQ ID NO: 23), GSGRSA (SEQ ID NO: 21) and GSGKSA (SEQ ID NO: 22).

29. The method of claim 24, wherein the protein overexpressed on the surface of a cell is a receptor.

30. The method of claim 24, wherein the cell is a cancer cell.

31. The method of claim 24, wherein the heterologous polypeptide comprises a cytokine.

32. The method of claim 24, wherein the heterologous polypeptide comprises a growth factor.

33. The method of claim 24, wherein the fusion protein is encoded by the nucleotide sequence set forth in SEQ ID NO: 2, 3, 4, 5, 6, 7, 8, 9, 10, 11, 12, or 13.

34. The method of claim 30, wherein the cancer is leukemia.

35. The method of claim 30, wherein the cancer is acute myelogenous leukemia.

36. The method of claim 31, wherein the heterologous polypeptide comprises GM-CSF.

37. The method of claim 31, wherein the heterologous polypeptide comprises IL-2.

38. The method of claim 32, wherein the heterologous polypeptide comprises EGF.

39. The method of claim 24, wherein the Diphtheria toxin fusion protein comprises:
(1) residues 1-388 of Diphtheria toxin, wherein the native furin cleavage site has been substituted for a cleavage site for a urokinase plasminogen activator; and
(2) GM-CSF.

40. A method of targeting a compound to a cell overexpressing a cytokine receptor or a growth factor receptor, the method comprising the steps of:
administering to the cell Diphtheria toxin fusion protein comprising
(1) residues 1-388 of Diphtheria toxin, wherein the native furin cleavage site has been substituted for a cleavage site for a matrix metalloproteinase or a plasminogen activator and wherein the Diphtheria toxin is cleaved by a matrix metalloproteinase or a plasminogen activator; and
(2) a heterologous polypeptide, wherein the heterologous polypeptide specifically binds to a cytokine receptor or a growth factor receptor.

41. The method of claim 40, wherein the cell also overexpresses a matrix metalloproteinase, a tissue plasminogen activator, or a urokinase plasminogen activator.

42. The method of claim 40, wherein the matrix metalloproteinase is selected from the group consisting of MMP-2 (gelatinase A), MMP-9 (gelatinase B) and membrane-typel MMP (MT1-MMP).

43. The method of claim 40, wherein the plasminogen activator is selected from the group consisting of t-PA and u-PA.

44. The method of claim 40, wherein the matrix metalloproteinase cleavage sites are GPLGMLSQ (SEQ ID NO: 19) and GPLGLWAQ SEQ ID NO: 20).

45. The method of claim 40, wherein the plasminogen activator cleavage site is selected from the group consisting of QRGRSA (SEQ ID NO: 23), GSGRSA (SEQ ID NO: 21) and GSGKSA (SEQ ID NO: 22).

46. The method of claim 40, wherein the cancer cell is a leukemia cell.

47. The method of claim 40, wherein the cancer cell is an acute myelogenous leukemia cell.

48. The method of claim 40, wherein the Diphtheria toxin fusion protein comprises
(1) residues 1-388 of Diphtheria toxin, wherein the native furin cleavage site has been substituted for a cleavage site for a urokinase plasminogen activator; and
(2) GM-CSF.

49. An isolated nucleic acid comprising the sequence set forth in any one of SEQ ID NOS: 2-18.


SEQUENCE LISTING (30 sequences)

SEQ ID NO: 1 - DNA, 1683 bp, Corynebacterium diphtheriae
  wild type Diphtheria toxin (DT)
gtgagcagaaaactgtttgcgtcaatcttaataggggcgctactggggataggggccccaccttcagcccatgcaggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga

SEQ ID NO: 2 - DNA, 1566 bp, Artificial Sequence
  Description of Artificial Sequence DTGM-L1 DT-GMCSF fusion protein in which native furin recognition cleavage site replaced by matrix metalloproteinase (MMP) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaccattaggaatgttgagtcaaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcaccagcacgatcgccaagcccaagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggctagccactacaagcagcactgccctccaaccccggaaacttcctgtgcgacccagactatcacctttgaaagtttcaaagagaacctgaaggactttctgcttgtcatcccctttgactgctgggagccagtacaggaagcttga

SEQ ID NO: 3 - DNA, 1566 bp, Artificial Sequence
  Description of Artificial Sequence DTGM-L2 DT-GMCSF fusion protein in which native furin recognition cleavage site replaced by matrix metalloproteinase (MMP) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaccattaggattatgggcacaaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcaccagcacgatcgccaagcccaagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggctagccactacaagcagcactgccctccaaccccggaaacttcctgtgcgacccagactatcacctttgaaagtttcaaagagaacctgaaggactttctgcttgtcatcccctttgactgctgggagccagtacaggaagcttga

SEQ ID NO: 4 - DNA, 1560 bp, Artificial Sequence
  Description of Artificial Sequence DTGM-U2 DT-GMCSF fusion protein in which native furin recognition cleavage site replaced by urokinase-type plasminogen activator (uPA) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaagtggaagatcagcaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcaccagcacgatcgccaagcccaagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggctagccactacaagcagcactgccctccaaccccggaaacttcctgtgcgacccagactatcacctttgaaagtttcaaagagaacctgaaggactttctgcttgtcatcccctttgactgctgggagccagtacaggaagcttga

SEQ ID NO: 5 - DNA, 1560 bp, Artificial Sequence
  Description of Artificial Sequence DTGM-U3 DT-GMCSF fusion protein in which native furin recognition cleavage site replaced by urokinase-type plasminogen activator (uPA) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaagtggaaaatcagcaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcaccagcacgatcgccaagcccaagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggctagccactacaagcagcactgccctccaaccccggaaacttcctgtgcgacccagactatcacctttgaaagtttcaaagagaacctgaaggactttctgcttgtcatcccctttgactgctgggagccagtacaggaagcttga

SEQ ID NO: 6 - DNA, 1341 bp, Artificial Sequence
  Description of Artificial Sequence DTEGF-L1 DT-EGF fusion protein in which native furin recognition cleavage site replaced by matrix metalloproteinase (MMP) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaccattaggaatgttgagtcaaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatgaattccgatagcgagtgtcctctgagtcacgatggttactgtctacatgacggcgtctgtatgtatattgaggctctagacaagtacgcgtgtaattgcgttgttggctacatcggtgagcgctgtcagtatcgagatctgaaatggtgggaacttagataa

SEQ ID NO: 7 - DNA, 1341 bp, Artificial Sequence
  Description of Artificial Sequence DTEGF-L2 DT-EGF fusion protein in which native furin recognition cleavage site replaced by matrix metalloproteinase (MMP) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaccattaggattatgggcacaaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatgaattccgatagcgagtgtcctctgagtcacgatggttactgtctacatgacggcgtctgtatgtatattgaggctctagacaagtacgcgtgtaattgcgttgttggctacatcggtgagcgctgtcagtatcgagatctgaaatggtgggaacttagataa

SEQ ID NO: 8 - DNA, 1335 bp, Artificial Sequence
  Description of Artificial Sequence DTEGF-U2 DT-EGF fusion protein in which native furin recognition cleavage site replaced by urokinase-type plasminogen activator (uPA) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaagtggaagatcagcaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatgaattccgatagcgagtgtcctctgagtcacgatggttactgtctacatgacggcgtctgtatgtatattgaggctctagacaagtacgcgtgtaattgcgttgttggctacatcggtgagcgctgtcagtatcgagatctgaaatggtgggaacttagataa

SEQ ID NO: 9 - DNA, 1335 bp, Artificial Sequence
  Description of Artificial Sequence DTEGF-U3 DT-EGF fusion protein in which native furin recognition cleavage site replaced by urokinase-type plasminogen activator (uPA) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaagtggaaaatcagcaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatgaattccgatagcgagtgtcctctgagtcacgatggttactgtctacatgacggcgtctgtatgtatattgaggctctagacaagtacgcgtgtaattgcgttgttggctacatcggtgagcgctgtcagtatcgagatctgaaatggtgggaacttagataa

SEQ ID NO: 10 - DNA, 1581 bp, Artificial Sequence
  Description of Artificial Sequence DTIL2-L1 DT-IL2 fusion protein in which native furin recognition cleavage site replaced by matrix metalloproteinase (MMP) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaccattaggaatgttgagtcaaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 11 - DNA, 1581 bp, Artificial Sequence
  Description of Artificial Sequence DTIL2-L2 DT-IL2 fusion protein in which native furin recognition cleavage site replaced by matrix metalloproteinase (MMP) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaccattaggattatgggcacaaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 12 - DNA, 1575 bp, Artificial Sequence
  Description of Artificial Sequence DTIL2-U2 DT-IL2 fusion protein in which native furin recognition cleavage site replaced by urokinase-type plasminogen activator (uPA) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaagtggaagatcagcaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 13 - DNA, 1575 bp, Artificial Sequence
  Description of Artificial Sequence DTIL2-U3 DT-IL2 fusion protein in which native furin recognition cleavage site replaced by urokinase-type plasminogen activator (uPA) recognition cleavage site
atgggcgccgacgacgtcgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatggaagtggaaaatcagcaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctcccgggcataaaacgaggcctcatatggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 14 - DNA, 22 bp, Artificial Sequence
  Description of Artificial Sequence universal 5′ T7 promoter primer (5′ primer for DT constructs)
gtaatacgactcactatagggc

SEQ ID NO: 15 - DNA, 61 bp, Artificial Sequence
  Description of Artificial Sequence U2 3′ mutagenic PCR primer for U2 constructs
gatttatgcatgacaatgagctacctgctgatcttccacttccatttcctgcacaggcttg

SEQ ID NO: 16 - DNA, 61 bp, Artificial Sequence
  Description of Artificial Sequence U3 3′ mutagenic PCR primer for U3 constructs
gatttatgcatgacaatgagctacctgctgattttccacttccatttcctgcacaggcttg

SEQ ID NO: 17 - DNA, 67 bp, Artificial Sequence
  Description of Artificial Sequence L1 3′ mutagenic PCR primer for L1 constructs
gatttatgcatgacaatgagctaccttgactcaacattcctaatggtccatttcctgcacaggcttg

SEQ ID NO: 18 - DNA, 67 bp, Artificial Sequence
  Description of Artificial Sequence L2 3′ mutagenic PCR primer for L2 constructs
gatttatgcatgacaatgagctaccttgtgcccataatcctaatggtccatttcctgcacaggcttg

SEQ ID NO: 19 - protein, 8 aa, Artificial Sequence
  Description of Artificial Sequence matrix metalloproteinase (MMP) recognition cleavage site, MMP substrate octapeptide for L1 constructs
GPLGMLSQ

SEQ ID NO: 20 - protein, 8 aa, Artificial Sequence
  Description of Artificial Sequence matrix metalloproteinase (MMP) recognition cleavage site, MMP substrate octapeptide for L2 constructs
GPLGLWAQ

SEQ ID NO: 21 - protein, 6 aa, Artificial Sequence
  Description of Artificial Sequence urokinase plasminogen activator (uPA) recognition cleavage site, uPA favorite sequence, uPA substrate hexapeptide for U2 constructs
GSGRSA

SEQ ID NO: 22 - protein, 6 aa, Artificial Sequence
  Description of Artificial Sequence urokinase plasminogen activator (uPA) recognition cleavage site, uPA favorite sequence, uPA substrate hexapeptide for U3 constructs
GSGKSA

SEQ ID NO: 23 - protein, 6 aa, Artificial Sequence
  Description of Artificial Sequence tissue-type plasminogen activator (tPA) recognition cleavage site, tPA favorite sequence
QRGRSA

SEQ ID NO: 24 - protein, 16 aa, Artificial Sequence
  Description of Artificial Sequence DTGM-WT furin sensitive surface loop sequence
CAGNRVRRSVGSSLSC

SEQ ID NO: 25 - protein, 16 aa, Artificial Sequence
  Description of Artificial Sequence DTGM-U2 surface loop sequence cleaved by urokinase-type plasminogen activator (uPA)
CAGNGSGRSAGSSLSC

SEQ ID NO: 26 - protein, 16 aa, Artificial Sequence
  Description of Artificial Sequence DTGM-U3 surface loop sequence cleaved by urokinase-type plasminogen activator (uPA)
CAGNGSGKSAGSSLSC

SEQ ID NO: 27 - protein, 18 aa, Artificial Sequence
  Description of Artificial Sequence DTGM-L1 surface loop sequence cleaved by matrix metalloproteinase (MMP)
CAGNGPLGMLSQGSSLSC

SEQ ID NO: 28 - protein, 16 aa, Artificial Sequence
  Description of Artificial Sequence DTGM-Fu surface loop sequence cleaved only by furin
CAGNRAARSVGSSLSC

SEQ ID NO: 29 - protein, 9 aa, Artificial Sequence
  Description of Artificial Sequence plasminogen activator cleavage site, uPA and tPA physiological substrate sequence
PCPGRVVGG

SEQ ID NO: 30 - protein, 6 aa, Artificial Sequence
  Description of Artificial Sequence Diphtheria toxin (DT) cleavage sequence amino acids 163-170
RVRRSV